(19) United States
(12) Patent Application Publication
(10) Pub. No.: US 2021/0210190 A1
(43) Pub. Date: Jul. 8, 2021

(54) Diagnostic Method

(71) Applicant: Amazentis SA, Lausanne (CH)

(72) Inventors: Christopher L. Rinsch, Lausanne (CH); Anurag Singh, Lausanne (CH)

(21) Appl. No.: 17/089,448

(22) Filed: Nov. 4, 2020

(30) Foreign Application Priority Data
Nov. 4, 2019 (GB) 1916046.4

(51) Int. Cl. G16H 20/60; A61K 31/352; G16H 10/40; G16H 20/10

(52) U.S. Cl. CPC G16H 20/60; A61K 31/352; G01N 33/49; G16H 20/10; G01N 2800/52; G16H 10/40

(57) ABSTRACT

The current application relates to personalised nutrition methods, particularly to methods of determining whether a human subject would benefit from taking a urolithin supplement and methods for determining the treatment dose of a urolithin supplement for a human subject. Particularly methods comprising determining the level of urolithin or a urolithin conjugate in a biological fluid, such as a dried whole blood spot sample, a dried plasma spot, a m spot sample or a urine sample The current application also relates to systems for presenting whether a human subject would benefit from taking a urolithin supplement and for presenting the treatment dose of a urolithin supplement for a human subject. The current application also relates to computer implementation of methods of the invention.

### RELATED APPLICATIONS

This application claims the benefit of priority to GB Patent Application No. 1916046.4, filed Nov. 4, 2019.

### FIELD

The current application relates to personalised nutrition methods, particularly to methods of determining whether a human subject would benefit from taking a urolithin supplement and methods for determining the treatment dose of a urolithin supplement for a human subject. Particularly methods comprising determining the level of urolithin or a urolithin conjugate in a biological fluid, such as a dried whole blood spot sample, or a dried plasma spot or serum spot sample or a urine sample. The current application also relates to systems for presenting whether a human subject would benefit from taking a urolithin supplement and for presenting the treatment dose of a urolithin supplement for a human subject. The current application also relates to computer implementation of methods of the invention.

### BACKGROUND

Urolithins have been proposed as treatments for a variety of conditions related to inadequate mitochondrial activity, including obesity, reduced metabolic rate, metabolic syndrome, diabetes mellitus, cardiovascular disease, hyperlipidaemia, neurodegenerative diseases, cognitive disorders, mood disorders, stress, and anxiety disorders; for weight management, or to increase muscle performance or mental performance, See WO2012/088519 (Amazentis SA). In WO2007/127263 (The Regents of the University of California), the use of urolithins for the treatment of various neoplastic diseases is described.

International patent publication WO2014/004902 (derived from application PCT/US2013/48310) discloses a method of increasing autophagy, including specifically mitophagy, in a cell, comprising contacting a cell with an effective amount of a urolithin or a pharmaceutically acceptable salt thereof, thereby increasing autophagy, including specifically mitophagy, in the cell. Administration may be to a subject having a disease or condition selected from metabolic stress, cardiovascular disease, endothelial cell dysfunction, sarcopenia, muscle degenerative disease, Duchenne muscular dystrophy, alcoholic liver disease, non-alcoholic fatty liver disease, drug-induced liver or muscle injury, al-antitrypsin deficiency, ischemia/reperfusion injury, inflammation, aging of the skin, inflammatory bowel disease, Crohn's disease, obesity, metabolic syndrome, type II diabetes mellitus, hyperlipidaemia, osteoarthritis, neurodegenerative disease, Alzheimer's disease, Huntington's disease, Parkinson's disease, amyotrophic lateral sclerosis, age-related macular degeneration, mitochondrial diseases (including for example poor growth, loss of muscle coordination, muscle weakness, visual problems, hearing problems, heart disease, liver disease, kidney disease, gastrointestinal disorders, respiratory disorders, neurological problems, autonomic dysfunction, learning disabilities, and dementia as a result of mitochondrial disease), muscle diseases; cancer, cognitive disorder, stress, and mood disorder.

In particular, urolithins have been proposed as treatments for muscle-related pathological conditions. Muscle-related pathological conditions include myopathies and neuromuscular diseases. Examples of such conditions include Duchenne muscular dystrophy, acute sarcopenia, for example muscle atrophy and/or cachexia, for example associated with burns, bed rest, limb immobilization, or major thoracic, abdominal, neck and/or orthopaedic surgery, amyotrophic lateral sclerosis and multiple sclerosis. Age-related muscle-loss is an especially prevalent condition. Cachexia due to prolonged immobilization or other diseases, for example cancer, are other conditions that are often characterised by poor muscle performance.

Effective muscle function and physical performance is important for having a high quality of life at all ages in healthy individuals as well as in those individuals suffering from a disease, especially the elderly. Improved muscle performance is of particular interest to athletes. For example, an increase in muscular contraction strength, increase in amplitude of muscle contraction, or shortening of muscle reaction time between stimulation and contraction are all of benefit to individuals, especially athletes. For elderly suffering from age related decline in muscle function including muscle loss/wasting or individuals suffering from cachexia muscle wasting, an improvement in muscle and physical performance is important for basic aspects of daily functioning such as walking speed and distance they can walk unassisted.

Urolithins are natural gut metabolites, produced by the host gut microflora upon exposure to dietary precursors (such as ellagitannins) that are found in high quantities in certain fruits and nuts such as pomegranate, berries and walnuts. Urolithin production in the gut shows a large human inter-individual variability and this has been associated with differences in the colon microbiota [García-Villalba et al (2013) J. Agric. Food Chem., 2013, 61, 8797-8806]. Some studies have shown health benefits from the consumption of these polyphenol-rich fruits, the results across these studies are not consistent, possibly due to the large variation in the gut microbiota between individuals, which will result in different metabolite profiles greatly influencing their bio-potency. This raises another major challenge in designing optimal dietary intervention and limits the interpretation of studies investigating health aspects of the consumption of polyphenol-rich fruits. [for example, see Tomás-Barberán F A et al (2014) J Agric Food Chem. 62(28):6535-8 and González-Sarrías et al (2017) Mol Nutr Food Res.61(5). doi: 10.1002/mnfr.201600830]. Therefore, there is a need to develop assays to determine which individuals would benefit from taking a urolithin and what dose would be required. The present application addresses this need.

### DESCRIPTION OF THE INVENTION

One embodiment of the present invention provides a method of determining whether a human subject will benefit from taking a urolithin supplement, comprising the steps of analysing a sample of a biological fluid collected from the subject, ascertaining whether the biological fluid contains a urolithin or a urolithin conjugate, then determining whether the subject is one who will benefit from ingesting a urolithin supplement from the level of the urolithin or urolithin conjugate in the biological fluid, for example, wherein the sample of biological fluid is selected from urine or blood and in the case of blood is analysed in the form of a dried blood spot.

In one embodiment of the invention, the urolithin conjugate is urolithin A glucuronide and the biological fluid is a dried blood spot and wherein if the level of urolithin A glucuronide is under 100 ng/mL, then the subject is one who will benefit from a dose of a urolithin supplement. In a further embodiment, if the level urolithin A glucuronide is under 50 ng/mL, then the subject is one who will benefit from a dose of a urolithin supplement. In a further embodiment, if the level urolithin A glucuronide is under 25 ng/mL, then the subject is one who will benefit from a dose of a urolithin supplement.

In a further embodiment of the invention the urolithin conjugate is urolithin A glucuronide and the biological fluid is urine and wherein if the level of urolithin A glucuronide is under 50,000 ng/mL, then the subject is one who will benefit from a dose of a urolithin supplement. In a further embodiment, if the level urolithin A glucuronide is under 25,000 ng/mL, then the subject is one who will benefit from a dose of a urolithin supplement. In a further embodiment, if the level urolithin A glucuronide is under 10,000 ng/mL, then the subject is one who will benefit from a dose of a urolithin supplement.

In a further embodiment of the invention, there is provided a method for determining a treatment dose of a urolithin supplement for a human subject, comprising
a) Measuring the level of the urolithin or a urolithin conjugate in a biological fluid; and
b) Determining the treatment dose of the urolithin supplement from the level of urolithin or urolithin conjugate in the biological fluid sample
   wherein the sample of biological fluid is selected from urine or blood and in the case of blood is analysed in the form of a dried blood spot.

In a one embodiment, when determining a treatment dose the urolithin conjugate is urolithin A glucuronide and the biological fluid is a dried blood spot and:
a) if the level of urolithin A glucuronide is less than 5 ng/mL then the treatment dose of urolithin supplement is 1000 mg per day or 1500 mg per day, for example 1000 mg per day;
b) if the level of urolithin A glucuronide is between 5 ng/mL and 50 ng/mL then the treatment dose of urolithin supplement is 500 mg per day; and
c) if the level of urolithin A glucuronide is between 50 ng/mL and 100 ng/mL the treatment dose of urolithin supplement is 250 mg/day.

In a further embodiment, when determining a treatment dose the urolithin conjugate is urolithin A glucuronide and the biological fluid is urine and
a) if the level of urolithin A glucuronide is less than 10,000 ng/mL then the treatment dose of urolithin supplement is 1500 mg or 1000 mg per day, for example 1000 mg per day;
b) if the level of urolithin A glucuronide is between 10,000 ng/mL and 25,000 ng/mL then the treatment dose of urolithin supplement is 500 mg per day; and
c) if the level of urolithin A glucuronide is between 25,000 ng/mL and 50,000 ng/mL the treatment dose of urolithin supplement is 250 mg/day.

In a further embodiment of the invention, there is provided a method for determining whether a human subject will benefit from taking a urolithin supplement, comprising
a) Measuring the level of a urolithin or a urolithin conjugate in a first biological fluid sample, collected prior to administration of a test dose of a urolithin or a urolithin precursor;
b) Administering a test dose of a urolithin of a urolithin precursor, for example pomegranate juice;
c) Measuring the level of the urolithin or a urolithin conjugate in a second biological fluid sample; and
d) Determining whether the subject is one who will benefit from ingesting a urolithin supplement from the increase in level of urolithin or urolithin conjugate in the biological fluid sample;
   wherein the sample of biological fluid is selected from urine or blood and in the case of blood is analysed in the form of a dried blood spot.; and
   for example,
(i) wherein a urolithin is administered, the urolithin conjugate is urolithin A glucuronide and the biological fluid is a dried drug spot, and wherein if the level of urolithin glucuronide is above 5 ng/mL and under 100 ng/mL, then the subject is one who will benefit from a dose of a urolithin supplement; or
(ii) wherein a urolithin precursor is administered, the urolithin conjugate is urolithin A glucuronide and the biological fluid is a dried drug spot and wherein if the level of urolithin glucuronide is under 100 ng/mL, then the subject is one who will benefit from a dose of a urolithin supplement; or
(iii) wherein a urolithin is administered, the urolithin conjugate is urolithin A glucuronide and the biological fluid is urine, and wherein if the level of urolithin glucuronide is above 5,000 ng/mL and under 50,000 ng/mL, then the subject is one who will benefit from a dose of a urolithin supplement; or
(iv) wherein a urolithin precursor is administered, the urolithin conjugate is urolithin A glucuronide and the biological fluid is urine and wherein if the level of urolithin glucuronide is under 50,000 ng/mL, then the subject is one who will benefit from a dose of a urolithin supplement.

A biological sample may be collected at a time after administration of a urolithin or urolithin precursor, when a urolithin or urolithin conjugate can be detected in the biological sample. For example, the sample, may be collected 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47 or 48 hours after administration of a urolithin or urolithin precursor. For example, 6-8 hours, 6-10 hours, 6-12 hours, 6-18 hours, 6-24 hours, 6-30 hours, 6-36 hours, 6-42 hours or 6-48 hours. For example, 12-18 hours, 12-24 hours, 12-30 hours, 12-36 hours, 12-42 hours or 12-48 hours. For example, 18-24 hours, 18-30 hours, 18-36 hours, 18-42 hours or 18-48 hours. For example, 24-30 hours, 24-36 hours, 24-42 hours or 24-48 hours. For example, 36-42 hours or 36-48 hours. In one embodiment, the biological fluid sample is collected either;
(a) between about 18 to about 30 hours after the administration of the urolithin or urolithin precursor; or
(b) at about 24 hours after the administration of the urolithin or the urolithin precursor;
(c) between about 6 hours and about 8 hours after administration of the urolithin or urolithin precursor.

In one embodiment the present invention provides a method of determining whether a human subject will benefit from taking a urolithin supplement, comprising the steps of analysing a dried blood spot sample taken from the subject, ascertaining whether the dried blood spot contains a urolithin or a urolithin conjugate, then determining whether the subject is one who will benefit from ingesting a urolithin supplement from the level of the urolithin or urolithin conjugate in the dried blood spot. In further embodiments of the invention, methods further comprise administering urolithin to the subject.

In one embodiment, the dried blood spot is a dried sample of whole blood.

In a further embodiment, the dried blood spot is a dried sample of plasma.

In a further embodiment, the dried blood spot is a dried sample of serum.

In one embodiment, the level of a urolithin, such as urolithin A, is measured. In another embodiment the level of a urolithin conjugate, such as urolithin A glucuronide, is measured. In a further embodiment the level of urolithin sulphate, such as urolithin A sulphate, is measured. In a further embodiment the level of urolithin A glucuronide is measured.

In one embodiment the human subject is one requiring medical treatment. In a further embodiment the human subject is one who is generally well but would benefit from taking a urolithin.

Dried blood spot samples are reconstituted in an aqueous fluid, such as water or a buffer or in a suitable solvent, such as methanol. Tests able to detect and measure the level of a urolithin or a urolithin conjugate in a dried blood spot aqueous sample are well known in the art.

In one embodiment when the level of urolithin glucuronide is under about 10 ng/mL, then the subject is one who will benefit from a dose of a urolithin supplement. In one embodiment when the level of urolithin glucuronide is under about 20 ng/mL, then the subject is one who will benefit from a dose of a urolithin supplement. In one embodiment when the level of urolithin glucuronide is under about 30 ng/mL, then the subject is one who will benefit from a dose of a urolithin supplement. In one embodiment when the level of urolithin glucuronide is under about 40 ng/mL, then the subject is one who will benefit from a dose of a urolithin supplement. In one embodiment when the level of urolithin glucuronide is under about 50 ng/mL, then the subject is one who will benefit from a dose of a urolithin supplement. In one embodiment when the level of urolithin glucuronide is under about 60 ng/mL, then the subject is one who will benefit from a dose of a urolithin supplement. In one embodiment when the level of urolithin glucuronide is under about 70 ng/mL, then the subject is one who will benefit from a dose of a urolithin supplement. In one embodiment when the level of urolithin glucuronide is under about 80 ng/mL, then the subject is one who will benefit from a dose of a urolithin supplement. In a further embodiment, when the level of urolithin glucuronide is under about 90 ng/mL, then the subject is one who will benefit from a dose of a urolithin supplement. In a further embodiment, when the level of urolithin glucuronide is under about 100 ng/mL, then the subject is one who will benefit from a dose of a urolithin supplement. In a further embodiment, when the level of urolithin glucuronide is under about 110 ng/mL, then the subject is one who will benefit from a dose of a urolithin supplement. In a further embodiment, when the level of urolithin glucuronide is under about 120 ng/mL, then the subject is one who will benefit from a dose of a urolithin supplement. In a further embodiment, when the level of urolithin glucuronide is under about 130 ng/mL, then the subject is one who will benefit from a dose of a urolithin supplement. In a further embodiment, when the level of urolithin glucuronide is under about 140 ng/mL, then the subject is one who will benefit from a dose of a urolithin supplement. In a further embodiment, when the level of urolithin glucuronide is under about 150 ng/mL, then the subject is one who will benefit from a dose of a urolithin supplement.

In addition to providing information as to whether a human subject would benefit from taking a urolithin supplement, the level of urolithin or a urolithin conjugate can also provide information as to a suitable treatment dose for a human subject. Therefore, according to a further aspect of the invention there is provided a method for determining a treatment dose of a urolithin supplement for a human subject, comprising

a) Measuring the level of urolithin or a urolithin conjugate in a dried blood spot sample; and

b) Determining the treatment dose of the urolithin supplement from the level of urolithin or urolithin conjugate in the dried blood spot sample.

In a further embodiment, the invention provides a method of administering a urolithin to a human subject, comprising:

a) Measuring the level of urolithin or a urolithin conjugate in a dried blood spot sample;

b) Determining the treatment dose of the urolithin supplement from the level of urolithin or urolithin conjugate in the dried blood spot sample; and

c) administering a urolithin to the subject.

In one embodiment the level of urolithin or a urolithin conjugate in a dried blood spot sample, may be used with a continuous scale such that for a urolithin or urolithin conjugate amount between 10 ng/mL to 100 ng/day would indicate a treatment dose between 100 mg/day to 1500 mg/day, for example, 100 mg/day to 1000 mg/day. In a further embodiment, the treatment dose would be selected in a series of bands of the amount of urolithin or a urolithin conjugate indicating a treatment dose of a unit dosage of urolithin. For example,

a) if the level of urolithin glucuronide is less than 10 ng/mL then the treatment dose of urolithin supplement is 1000 mg per day;

b) if the level of urolithin glucuronide is between 10 ng/mL and 50 ng/mL then the treatment dose of urolithin supplement is 500 mg per day; and

c) if the level of urolithin glucuronide is between 50 ng/mL and 100 ng/mL the treatment dose of urolithin supplement is 250 mg/day.

For example,

a) if the level of urolithin glucuronide is less than 5 ng/mL then the treatment dose of urolithin supplement is 1000 mg per day;

b) if the level of urolithin glucuronide is between 5 ng/mL and 50 ng/mL then the treatment dose of urolithin supplement is 500 mg per day; and

c) if the level of urolithin glucuronide is between 50 ng/mL and 100 ng/mL the treatment dose of urolithin supplement is 250 mg/day.

In addition to measuring the amount of urolithin or a urolithin conjugate in a dried blood sample, in isolation, the invention also provides methods wherein a source of urolithin is ingested by the human subject. Such a source of urolithin may comprise a unit dosage form comprising urolithin or a urolithin precursor. Therefore, in a further embodiment of the invention there is provided a method for determining whether a human subject will benefit from taking a urolithin supplement, comprising

a) Measuring the level of a urolithin or a urolithin conjugate in a first dried blood spot sample, collected prior to administration of a test dose of urolithin;

b) Administering a test dose of a urolithin;

c) Measuring the level of the urolithin or a urolithin conjugate in a second dried blood spot sample; and

d) Determining whether the subject is one who will benefit from ingesting a urolithin supplement from the increase in level of urolithin or urolithin conjugate in the dried blood spot sample.

In a further embodiment of the invention there is provided a method for administering a urolithin to a human subject, comprising

a) Measuring the level of a urolithin or a urolithin conjugate in a first dried blood spot sample, collected prior to administration of a test dose of urolithin;

b) Administering a test dose of a urolithin;

c) Measuring the level of the urolithin or a urolithin conjugate in a second dried blood spot sample;

d) Determining whether the subject is one who will benefit from ingesting a urolithin supplement from the increase in level of urolithin or urolithin conjugate in the dried blood spot sample; and

e) Administering a urolithin to the subject.

For the avoidance of doubt, for any method of the invention, which comprises administration of a urolithin or a urolithin precursor, the method may be conducted without taking a first biological sample, for example, urine or blood and only taking a biological sample for urolithin/urolithin conjugate measurement after administration of the urolithin or urolithin precursor. Decisions on benefit for the administration of a urolithin supplement or dose of said supplement can then be made based on the level of the urolithin or urolithin conjugate in the sample taken after administration of the urolithin or urolithin precursor.

The level of urolithin and/or urolithin conjugate in the dried blood sample after ingesting a urolithin would depend on the efficiency of uptake and metabolism of the urolithin or urolithin precursor in the human subject and the amount of a urolithin ingested. In one embodiment, if the level of urolithin glucuronide is above 10 ng/mL and under 150 ng/mL, then the subject is one who will benefit from a dose of a urolithin supplement. In a further embodiment if the level of urolithin glucuronide is under 150 ng/mL, then the subject is one who will benefit from a dose of a urolithin supplement. In a further embodiment, if the level of urolithin glucuronide is above 5 ng/mL and under 100 ng/mL, then the subject is one who will benefit from a dose of a urolithin supplement. In a further embodiment if the level of urolithin glucuronide is under 100 ng/mL, then the subject is one who will benefit from a dose of a urolithin supplement. In a further embodiment, the level of urolithin glucuronide is correlated with a treatment dose of 250 mg, 500 mg, 1000 mg or 1500 mg as defined above.

The level of urolithin and/or a urolithin conjugate can be measured by a method known to a person skilled in the art, for example, high performance liquid chromatography linked to mass spectrometry (HPLC/MS).

Prior to ingesting a urolithin supplement, the level of a urolithin or a urolithin conjugate in dried blood spot will predominantly depend on whether a subject has the required gut microbiome to metabolise a urolithin precursor to a urolithin. However, it will also depend on the diet of the said human subject. In a further embodiment, a human subject can be given a urolithin precursor prior to the collecting of a dried blood spot sample. Therefore, according to a further embodiment of the invention there is provided a method for determining whether a human subject will benefit from taking a urolithin supplement, comprising
a) Measuring the level of a urolithin or a urolithin conjugate in a first dried blood spot sample, collected prior to administration of a urolithin precursor, for example, pomegranate juice;
b) Administering the urolithin precursor;
c) Measuring the level of the urolithin or a urolithin conjugate in a second dried blood spot sample; and
d) Determining whether the subject is one who will benefit from ingesting a urolithin supplement from the increase in the level of urolithin or urolithin conjugate in the second dried blood spot sample compared to the first dried blood spot sample.

Therefore, according to a further embodiment of the invention there is provided a method for administering a urolithin to a human subject, comprising
a) Measuring the level of a urolithin or a urolithin conjugate in a first dried blood spot sample, collected prior to administration of a urolithin precursor, for example, pomegranate juice;
b) Administering the urolithin precursor;
c) Measuring the level of the urolithin or a urolithin conjugate in a second dried blood spot sample;
d) Determining whether the subject is one who will benefit from ingesting a urolithin supplement from the increase in the level of urolithin or urolithin conjugate in the second dried blood spot sample compared to the first dried blood spot sample; and
e) Administering a urolithin to the subject.

The level of urolithin and/or urolithin conjugate in the dried blood spot sample after ingesting a urolithin precursor would depend on the efficiency of metabolism of the urolithin precursor in the human subject and the amount of a urolithin precursor ingested. In one embodiment, if the level of urolithin glucuronide is above 10 ng/mL and under 150 ng/mL, then the subject is one who will benefit from a dose of a urolithin supplement. In a further embodiment if the level of urolithin glucuronide is under 150 ng/mL, then the subject is one who will benefit from a dose of a urolithin supplement. In a further embodiment, if the level of urolithin glucuronide is above 5 ng/mL and under 100 ng/mL, then the subject is one who will benefit from a dose of a urolithin supplement. In a further embodiment if the level of urolithin glucuronide is under 100 ng/mL, then the subject is one who will benefit from a dose of a urolithin supplement. In a further embodiment the level of urolithin glucuronide is correlated with a treatment dose of 250 mg, 500 mg, 1000 mg or 1500 mg as defined above.

In any embodiment of the invention, the dose of the urolithin test dose can be any dose of urolithin which produces a measurable amount of a urolithin or a urolithin conjugate in the dried blood spot or urine sample. For example, the test dose may selected from 50 mg, 100 mg, 250 mg, 500 mg or 1000 mg, such as 250 mg, 500 mg or 1000 mg, such as 250 mg or 500 mg. In one embodiment, the test dose is selected from 250 mg, 500 mg or 1000 mg.

The dried blood spot sample may be collected at any time after the ingestion of the urolithin or urolithin precursor when a urolithin or urolithin conjugate is detectable in the dried blood. For example, any time between about 2 hours and about 72 hours after ingestion of the urolithin or urolithin precursor, such between about 2 hours and about 60 hours, such as between about 2 hours and about 48 hours, such as between about 4 hours and about 48 hours, such as between about 4 hours and about 36 hours, such as between about 4 hours and about 24 hours after ingestion of the urolithin supplement. In one embodiment, about 4 hours, about 6 hours, about 8 hours, about 10 hours, about 12 hours or about 24 hours from ingestion of the urolithin or urolithin supplement, for example, about 6 hours, about 8 hours, or about 24 hours.

Surprisingly, we have observed a correlation between the ability of subjects to metabolise a urolithin precursor, such as pomegranate juice, to form urolithin A and the health of the subject's microbiome. Therefore, the level of urolithin or a urolithin conjugate in a sample of dried blood or urine is a biomarker of a healthy gut microbiome. The health of a subject's microbiome is measured by the diversity and richness of the gut microflora.

As mentioned above the level of a urolithin or a urolithin conjugate in dried blood spot will predominantly depend on whether a subject has the required gut microflora to metabolise a urolithin precursor to a urolithin. Therefore, the level of urolithin or a urolithin conjugate in the dried blood is a biomarker of a healthy gut microbiome. Therefore, according to a further aspect of the invention, there is provided a method of determining the health of an individual's gut microbiome comprising the steps of analysing a dried blood spot sample collected from the subject, ascertaining whether the dried blood spot contains a urolithin or a urolithin conjugate, as an indicator of the health of the individual's gut microbiome.

According to a further embodiment of the invention there is provided a method of determining the health of a subject's gut microbiome comprising the steps of analysing a sample of biological fluid collected from the subject, ascertaining whether the biological fluid, contains a urolithin or a urolithin conjugate, as an indicator of the health of the subject's gut microbiome; wherein the sample of biological fluid is selected from urine or blood and in the case of blood is analysed in the form of a dried blood spot.

According to a further aspect of the invention, there is provided a method of determining the health of an subject's gut microbiome comprising the steps of analysing a sample of biological fluid collected from the subject, ascertaining the level of urolithin or a urolithin conjugate in the biological fluid, and optionally collecting a stool sample for microbiome analysis, as an indicator of the health of the subject's gut;

wherein the sample of biological fluid is selected from urine or blood and in the case of blood is analysed in the form of a dried blood spot.

According to a further aspect of the invention, there is provided a method of determining the health of an individual's gut microbiome comprising the steps of analysing a dried blood spot sample collected from the subject, ascertaining the level of urolithin or a urolithin conjugate in the dried blood spot, as an indicator of the health of the individual's gut microbiome.

According to a further aspect of the invention, there is provided a method of determining the health of a subject's gut microbiome comprising the steps of analysing a dried blood spot sample collected from the subject, ascertaining the level of urolithin or a urolithin conjugate in the dried blood spot, as an indicator of the health of the subject's gut microbiome.

According to a further aspect of the invention, there is provided a method of determining the health of an subject's gut microbiome comprising the steps of analysing a dried blood spot sample collected from the subject, ascertaining the level of urolithin or a urolithin conjugate in the dried blood spot, and optionally collecting a stool sample for microbiome analysis, as an indicator of the health of the subject's gut microbiome.

According to a further aspect of the invention, there is provided a method of determining the health of an subject's gut microbiome comprising the steps of analysing a sample of urine collected from the subject, ascertaining whether the urine contains a urolithin or a urolithin conjugate, as an indicator of the health of the subject's gut microbiome.

According to a further aspect of the invention, there is provided a method of determining the health of a subject's gut microbiome comprising the steps of analysing a sample of urine collected from the subject, ascertaining the level of urolithin or a urolithin conjugate in the urine, and optionally collecting a stool sample for microbiome analysis, as an indicator of the health of the subject's gut microbiome.

In one embodiment, the method of determining the health of a subject's microbiome comprises administration of a urolithin precursor, such as pomegranate juice, prior to measuring the level of a urolithin or a urolithin conjugate, in a biological sample;

wherein the sample of biological fluid is selected from urine or blood and in the case of blood is analysed in the form of a dried blood spot.

In a further embodiment, if the subject is a low producer or a high producer for the conversion of a urolithin precursor to a urolithin, for example, urolithin A then the subject has a rich microbiome.

In a further embodiment, after the administration of a urolithin precursor, such as pomegranate juice a low producer is a subject who has 5-100 ng/mL urolithin A glucuronide as measured in a dry blood spot sample and a high producer has greater than 100 ng/mL of a urolithin A glucuronide.

In a further embodiment, after the administration of a urolithin precursor, such as pomegranate juice a low producer is a subject who has 5,000 g/mL to 25,000 ng/mL urolithin A glucuronide as measured in a urine and a high producer has greater than 50,000 ng/mL, for example, greater than 25,000 ng/mL of a urolithin A glucuronide in urine.

It is believed the higher the production or urolithin A or urolithin A glucuronide, the richer the microbiome of the subject.

In one embodiment the measurement of the urolithin or urolithin conjugate as a measure of microbiome health is measured at about 6 to about 8 hours after administration of a urolithin precursor or about 24 hours after administration of a urolithin precursor.

In a further aspect of the invention, there is provided a method for determining a treatment dose of a urolithin supplement for a human subject, comprising
a) Measuring the level of urolithin or a urolithin conjugate in a dried blood spot sample; and
b) Determining the treatment dose of the urolithin supplement from the level of urolithin or urolithin conjugate in the dried blood spot sample.

In a further embodiment, there is provided a method of estimating the concentration of a urolithin or urolithin conjugate in blood, plasma or serum, comprising:
(a) calculating the level of urolithin or a urolithin conjugate in a dry blood spot;
(b) converting the level to a concentration of urolithin using the volume of the blood used to make the dry blood spot
(c) multiplying the concentration by a factor of about 3.5 to about 4.5, for example, about 3.7 to about 4.3, for example about 3.5 to about 4.0, for example about 3.8 to about 4.2, for example about 3.7 to 3.9.

A further embodiment of the present invention provides a method of determining whether a human subject will benefit from taking a urolithin supplement, comprising the steps of analysing a sample of urine taken from the subject, ascertaining whether the urine contains a urolithin or a urolithin conjugate, then determining whether the subject is one who will benefit from ingesting a urolithin supplement from the level of the urolithin or urolithin conjugate in the urine.

In further embodiment the present invention provides a method of administering a urolithin to a human subject, comprising the steps of analysing a sample of urine taken from the subject, ascertaining whether the urine contains a urolithin or a urolithin conjugate, then determining whether the subject is one who will benefit from ingesting a urolithin supplement from the level of the urolithin or urolithin conjugate in the urine, and administering a urolithin to the subject.

In one embodiment, the level of a urolithin is measured in methods of the invention. In another embodiment, the level of a urolithin conjugate is measured in methods of the invention. In a further embodiment, the level of urolithin sulphate is measured in methods of the invention. In a further embodiment, the level of urolithin glucuronide is measured in methods of the invention. For example, a urolithin A or a urolithin A conjugate, such as urolithin A glucuronide.

In one embodiment, the human subject is one requiring medical treatment. In a further embodiment, the human subject is one who is generally well but would benefit from taking a urolithin.

Tests able to measure the level of urolithin or a urolithin conjugate in a urine sample are well known in the art.

In a further embodiment, when the level of urolithin glucuronide is under about 10,000 ng/mL, then the subject is one who will benefit from a dose of a urolithin supplement. In a further embodiment, when the level of urolithin glucuronide is under about 20,000 ng/mL, then the subject is one who will benefit from a dose of a urolithin supplement. In a further embodiment, when the level of urolithin glucuronide is under about 30,000 ng/mL, then the subject is one who will benefit from a dose of a urolithin supplement. In a further embodiment, when the level of urolithin glucuronide is under about 40,000 ng/mL, then the subject is one who will benefit from a dose of a urolithin supplement. In a further embodiment, when the level of urolithin glucuronide is under about 50,000 ng/mL, then the subject is one who will benefit from a dose of a urolithin supplement. In a further embodiment, when the level of urolithin glucuronide is under about 60,000 ng/mL, then the subject is one who will benefit from a dose of a urolithin supplement.

In addition to providing information as to whether a human subject would benefit from taking a urolithin supplement, the level of urolithin or a urolithin conjugate can also provide information as to a suitable treatment dose for a human subject. Therefore, according to a further aspect of the invention there is provided a method for determining a treatment dose of a urolithin supplement for a human subject, comprising

a) Measuring the level of a urolithin or a urolithin conjugate in a urine sample; and

b) Determining the treatment dose of the urolithin supplement from the level of urolithin or urolithin conjugate in the urine sample.

In a further aspect of the invention there is provided a method for administering a urolithin supplement to a human subject, comprising

a) Measuring the level of a urolithin or a urolithin conjugate in a urine sample;

b) Determining the treatment dose of the urolithin supplement from the level of urolithin or urolithin conjugate in the urine sample; and

c) Administering the treatment dose of urolithin to the human subject.

In one embodiment the level of urolithin or a urolithin conjugate in a urine sample, may be used with a continuous scale such that for a urolithin or urolithin conjugate amount between 10,000 ng/mL to 50,000 ng/day would indicate a treatment dose between 100 mg/day to 1500 mg/day or 100 mg/day to 1000 mg/day. In a further embodiment, the treatment dose would be selected in a series of bands of the amount of urolithin or a urolithin conjugate indicating a treatment dose of a unit dosage of urolithin. For example,

a) if the level of urolithin glucuronide is less than about 10,000 ng/mL then the treatment dose of urolithin supplement is 1500 mg or 1000 mg per day, for example, 100 mg per day;

b) if the level of urolithin glucuronide is between about 10,000 ng/mL and about 25,000 ng/mL then the treatment dose of urolithin supplement is 500 mg per day; and

c) if the level of urolithin glucuronide is between about 25,000 ng/mL and about 50,000 ng/mL the treatment dose of urolithin supplement is 250 mg/day.

In addition to measuring the amount of urolithin or a urolithin conjugate in a urine sample, in isolation, the invention also provides methods wherein a source of urolithin is ingested by the human subject. Such a source of urolithin may comprise a unit dosage form comprising urolithin or a urolithin precursor. Therefore, in a further embodiment of the invention there is provided a method for determining whether a human subject will benefit from taking a urolithin supplement, comprising

a) Optionally, measuring the level of a urolithin or a urolithin conjugate in a urine sample, collected prior to administration of a test dose of urolithin or a urolithin precursor;

b) Administering a test dose of a urolithin or a urolithin precursor;

c) Measuring the level of the urolithin or a urolithin conjugate in a urine sample, collected after administration of the test dose, for example, about 24 hours after administration of the test dose; and

d) Determining whether the subject is one who will benefit from ingesting a urolithin supplement from the level or increase in level of urolithin or urolithin conjugate in the urine sample.

In a further embodiment of the invention there is provided a method of administering a urolithin to a human, comprising

a) Optionally measuring the level of a urolithin or a urolithin conjugate in a urine sample, collected prior to administration of a test dose of urolithin or a urolithin precursor;

b) Administering a test dose of a urolithin or a urolithin precursor;

c) Measuring the level of the urolithin or a urolithin conjugate in a urine sample, collected after administration of the test dose, for example, about 24 hours after administration of the test dose;

d) Determining whether the subject is one who will benefit from ingesting a urolithin supplement from the level or increase in level of urolithin or urolithin conjugate in the urine sample; and

e) Administering a urolithin or urolithin precursor to the subject.

The level of urolithin and/or urolithin conjugate in the urine sample after ingesting a urolithin or a urolithin precursor would depend on the efficiency of uptake and metabolism of the urolithin or urolithin precursor in the human subject. In one embodiment, if the level of urolithin glucuronide is above 10,000 ng/mL and under 50,000 ng/mL, then the subject is one who will benefit from a dose of a urolithin supplement. In a further embodiment if the level of urolithin glucuronide is under 50,000 ng/mL, then the subject is one who will benefit from a dose of a urolithin supplement. In a further embodiment the level of urolithin glucuronide is correlated with a treatment dose of 250 mg, 500 mg, 1000 mg or 1500 mg as defined above.

The level of urolithin and/or a urolithin conjugate can be measured by a method known to a person skilled in the art, for example, high performance liquid chromatography linked to mass spectrometry (HPLC/MS).

Prior to ingesting a urolithin supplement, the level of a urolithin or a urolithin conjugate in urine will predominantly depend on whether a subject has the required gut microflora to metabolise a urolithin precursor to a urolithin. However, it will also depend on the diet of said human subject. In a further embodiment, a human subject can be given a urolithin precursor prior to the collecting of a urine sample. Therefore, according to a further embodiment of the invention there is provided a method for determining whether a human subject will benefit from taking a urolithin supplement, comprising
a) Measuring the level of a urolithin or a urolithin conjugate in a first urine sample, collected prior to administration of a urolithin precursor, for example, pomegranate juice;
b) Administering the urolithin precursor;
c) Measuring the level of the urolithin or a urolithin conjugate in a second urine sample; and
d) Determining whether the subject is one who will benefit from ingesting a urolithin supplement from the increase in the level of urolithin or urolithin conjugate in the second urine sample compared to the first urine sample.

In a further embodiment of the invention there is provided a method for administering a urolithin to a human subject, comprising
a) Measuring the level of a urolithin or a urolithin conjugate in a first urine sample, collected prior to administration of a urolithin precursor, for example, pomegranate juice;
b) Administering the urolithin precursor;
c) Measuring the level of the urolithin or a urolithin conjugate in a second urine sample;
d) Determining whether the subject is one who will benefit from ingesting a urolithin supplement from the increase in the level of urolithin or urolithin conjugate in the second urine sample compared to the first urine sample;
e) Administering a urolithin to the subject.

The level of urolithin and/or urolithin conjugate in the urine sample after ingesting a urolithin precursor would depend on the efficiency of metabolism of the urolithin precursor in the human subject. In one embodiment, if the level of urolithin glucuronide is above 5,000 ng/mL and under 50,000 ng/mL, then the subject is one who will benefit from a dose of a urolithin supplement. In a further embodiment if the level of urolithin glucuronide is under 50,000 ng/mL, then the subject is one who will benefit from a dose of a urolithin supplement. In a further embodiment, the level of urolithin glucuronide is correlated with a treatment dose of 250 mg, 500 mg, 1000 mg or 1500 mg as defined above.

The level of urolithin or a urolithin conjugate in urine after administration of a urolithin or a urolithin precursor can also be used to determine the treatment dose of a urolithin for the subject. In one embodiment the level of urolithin or a urolithin conjugate in a urine sample, may be used with a continuous scale such that for a urolithin or urolithin conjugate amount between 5,000 ng/mL to 50,000 ng/day would indicate a treatment dose between 100 mg/day to 1500 mg/day, such as 100 mg/day to 1000 mg/day. In a further embodiment, the treatment dose would be selected in a series of bands of the amount of urolithin or a urolithin conjugate indicating a treatment dose of a unit dosage of urolithin. For example,

a) if the level of urolithin glucuronide is less than about 10,000 ng/mL then the treatment dose of urolithin supplement is 1000 mg per day;

b) if the level of urolithin glucuronide is between about 10,000 ng/mL and about 25,000 ng/mL then the treatment dose of urolithin supplement is 500 mg per day; and

c) if the level of urolithin glucuronide is between about 25,000 ng/mL and about 50,000 ng/mL the treatment dose of urolithin supplement is 250 mg/day.

The dose of the urolithin test dose can be any dose of urolithin, which produces a measurable amount of a urolithin or a urolithin conjugate in the urine. For example, the test dose may selected from 50 mg, 100 mg, 250 mg, 500 mg or 1000 mg, such as 250 mg, 500 mg or 1000 mg, such as 250 mg or 500 mg.

The urine sample may be collected at any time after the ingestion of the urolithin or urolithin precursor when a urolithin or urolithin conjugate is detectable in the urine. For example, any time between about 2 hours and about 72 hours after ingestion of the urolithin or urolithin precursor, such between about 2 hours and about 60 hours, such as between about 2 hours and about 48 hours, such as between about 4 hours and about 48 hours, such as between about 4 hours and about 36 hours, such as between about 4 hours and about 24 hours after ingestion of the urolithin precursor. In one embodiment, about 4 hours, about 6 hours, about 8 hours, about 10 hours, about 12 hours, about 18 hours or about 24 hours from ingestion of the urolithin or urolithin precursor, for example, about 24 hours after ingestion of the urolithin or urolithin precursor.

In a further aspect of the invention, there is provided a method for determining a treatment dose of a urolithin supplement for a human subject, comprising
a) Measuring the level of the urolithin or a urolithin conjugate in a urine sample; and
b) Determining the treatment dose of the urolithin supplement from the level of urolithin or urolithin conjugate in the urine sample.

In a further aspect of the invention, there is provided a method for administering a urolithin to a human subject, comprising
a) Measuring the level of the urolithin or a urolithin conjugate in a urine sample;
b) Determining the treatment dose of the urolithin supplement from the level of urolithin or urolithin conjugate in the urine sample; and
c) Administering the treatment dose to the subject.

### Kits of the Invention

One consideration in the use of methods of the invention is making these accessible for the subject. One approach to this is the provision of a kit with all the materials required by the subject to collect the dried blood spot for the method and testing the dried blood spot. Therefore, in one embodiment of the invention, there is provided a kit for a method of determining whether a human subject will benefit from taking a urolithin supplement or for a method for determining a treatment dose of a urolithin supplement for a human subject, comprising:
(a) One of more fluid collection devices for collecting dried blood;
(b) Optionally a urolithin or a urolithin precursor;
(c) Instructions for taking the dried blood spot sample or dried blood samples; and
(d) Means for testing the dried blood spot sample(s) or means for getting the dried blood sample(s) tested (for example, a suitable postal envelope or postal container).

The fluid collection device may be any solid absorbent support suitable for collecting a whole blood sample, a plasma sample or a serum sample on which said blood or plasma or serum sample may be dried. Such devices can be stored for subsequent urolithin measurements.

In one embodiment, the filter collection device is a card. Such cards include filter paper, such as cellulose filter paper, for example, Whatman 903 filter paper or Whatman 903 sample collection cards (GE Healthcare, Little Chalfont, Buckinghamshire, UK) or a blood collection card from Perkin Elmer (Boston, Mass., USA).

Fluid collection cards also comprise cards, which allow separation of the components of a blood sample, for example, separating the red blood cells from plasma, such that measurement can be made in plasma. For example, the ADX100 blood card (Advance Dx Inc. Chicago, Ill., USA.) Further information, pertaining to the ADX blood card can be found in U.S. Pat. No. 8,062,608.

In a further embodiment, the filter collection device comprises an absorbent wand which is able to absorb whole blood, plasma or serum sample and allowed to dry. The tip of such a wand comprises a hydrophilic porous material for collecting the sample, such as a tip comprising a hydrophilic polymer material, for example, polyethylene. Examples of such devices include: the Mitra™ microsampler (Neoteryx, LLC., Torrance, Calif., USA). More information concerning the can be found in International patent application Number: WO 2013/067520.

In a further embodiment, the blood collection device comprises a sealable container, comprising an absorbent material and a desiccant, which once a whole blood or plasma or serum sample has been applied to the absorbent material, the container can be sealed for storage and shipping. Examples of such devices include: hemaspot HF™ (Spot on Sciences, San Francisco, Calif., USA). More information on the hemaspot HF™ device can be found in U.S. Pat. No. 9,759,640.

Blood samples are generally collected via a finger prick using a lance or similar sharp implement (preferably a sterile lance or sterile similar sharp implement). The blood sample consists of a number of drops of blood, for example, 3-4 drops, allowed to drip onto the fluid collection device or be absorbed onto the fluid collection device. Blood samples may also come from samples of blood, for example, sample of venous blood, obtained using a syringe or cannula.

As described above, method of the invention apply to dried spots of whole blood, dried spots of serum or dried spots of plasma. Therefore, prior to being dried samples of whole blood can be separated into plasma or serum, for example by centrifugation or coagulation.

A single dried blood spot sample may be taken or several dried blood spot samples at different times.

In a further embodiment, there is provided a kit for a method of determining whether a human subject will benefit from taking a urolithin supplement or for a method for determining a treatment dose of a urolithin supplement for a human subject, comprising:
(a) One of more fluid collection devices for collecting dried blood spots;
(b) Instructions for taking the dried blood spot sample or dried blood spot samples; and
(c) Means for testing the dried blood spot sample(s) or means for getting the dried blood spot sample(s) tested (for example, a suitable postal envelope or postal container).

In a further embodiment, there is provided a kit for a method of determining whether a human subject will benefit from taking a urolithin supplement or for a method for determining a treatment dose of a urolithin supplement for a human subject, comprising:
(d) One of more fluid collection devices for collecting dried blood;
(e) a urolithin or a urolithin precursor, for example, urolithin A;
(f) Instructions for taking the dried blood spot sample or dried blood spot samples; and
(g) Means for testing the dried blood spot sample(s) or means for getting the dried blood spot sample(s) tested (for example, a suitable postal envelope or postal container).

A further approach is the provision of a kit with all the materials required by the subject to collect the urine for the method and testing the urine. Therefore, in one embodiment of the invention, there is provided a kit for a method of determining whether a human subject will benefit from taking a urolithin supplement or for a method for determining a treatment dose of a urolithin supplement for a human subject, comprising:

(a) One of more containers for collecting urine;
(b) Optionally a urolithin or a urolithin precursor; and
(c) Instructions for taking the urine sample or urine samples.

In a further embodiment of the invention, there is provided a kit for a method of determining whether a human subject will benefit from taking a urolithin supplement or for a method for determining a treatment dose of a urolithin supplement for a human subject, comprising:
(a) One of more containers for collecting urine;
(b) Optionally a urolithin or a urolithin precursor;
(c) Instructions for taking the urine sample or urine samples; and
(d) Means for testing the urine sample(s) or means for transporting the urine sample(s) to a testing venue (for example, a suitable postal envelope or postal container).

Containers may be any container suitable for collecting a urine sample, for example, BD Vacutainer® plus urinalysis tubes (Becton Dickinson, Franklin Lakes, N.J., USA). Containers may be empty or contain reagents, such as lyophilised preservatives (such as boric acid, sodium formate and sodium borate) that maintain the sample integrity of urine samples at room temperature.

A single urine sample may be taken or several urine samples at different times.

In a further embodiment, there is provided a kit for a method of determining whether a human subject will benefit from taking a urolithin supplement or for a method for determining a treatment dose of a urolithin supplement for a human subject, comprising:
(a) One of more containers for collecting urine;
(b) a urolithin, for example, urolithin A or a urolithin precursor, for example, pomegranate juice;
(c) Instructions for taking the urine sample or urine samples; and
(d) Means for testing the urine sample(s) or means for transporting the urine sample(s) to a testing venue (for example, a suitable postal envelope or postal container).

### Systems of the Invention

In a further embodiment of the invention, there is provided a means for presenting relevant information concerning the methods of the invention. Relevant information includes status of the measurement of the level of the urolithin or urolithin conjugate in the dried blood spot or urine sample, the results of testing of the dried blood spot or urine sample, for example the level of the urolithin or urolithin conjugate in the dried blood spot or urine sample, or the treatment dose of a urolithin and/or the conclusion drawn from the level of urolithin or urolithin conjugate in the dried blood spot sample or urine sample, for example, whether a subject would benefit from taking a urolithin or urolithin supplement and/or the dose of a urolithin or urolithin supplement.

One approach to the presenting of the relevant information is through a user interface via a software program, for example, a mobile application, such a mobile application on a mobile phone or tablet device, a so called ‘app’. In one embodiment, the user interface is an internet user interface.

Therefore, according to a further embodiment of the invention, there is provided a system for presenting whether a human subject will benefit from taking a urolithin supplement, comprising the steps of:
a) analysing a sample of dried blood spot collected from the subject;
b) ascertaining the level of urolithin or a urolithin conjugate in the dried blood spot sample;
c) determining whether the subject is one who will benefit from ingesting a urolithin supplement from the level of urolithin in the dried blood spot; and
d) presenting on a user interface whether the subject is one who will benefit from a dose of urolithin supplement.

In a further embodiment of the invention, there is provided a system for presenting the treatment dose of a urolithin supplement for a human subject, comprising the steps of:
a) analysing a sample of dried blood spot collected from the subject;
b) ascertaining the level of a urolithin or a urolithin conjugate in the dried blood spot sample;
c) determining the treatment dose of urolithin supplement for the subject, from the level of urolithin or urolithin conjugate in the dried blood spot sample; and
d) presenting on a user interface the treatment dose of urolithin supplement for the subject.

In a further embodiment of the invention, there is provided a system for presenting whether a human subject will benefit from taking a urolithin supplement, comprising the steps of:
a) analysing a sample of urine collected from the subject;
b) ascertaining the level of urolithin or a urolithin conjugate in the urine sample;
c) determining whether the subject is one who will benefit from ingesting a urolithin supplement from the level of urolithin in the urine; and
d) presenting on a user interface whether the subject is one who will benefit from a dose of urolithin supplement.

In a further embodiment of the invention, there is provided a system for presenting the treatment dose of a urolithin supplement for a human subject, comprising the steps of:
a) analysing a sample of urine collected from the subject;
b) ascertaining the level of a urolithin or a urolithin conjugate in the urine sample;
c) determining the treatment dose of urolithin supplement for the subject, from the level of urolithin or urolithin conjugate in the urine sample; and
d) presenting on a user interface the treatment dose of urolithin supplement for the subject.

In a further embodiment of the invention, there is provided a system for presenting whether a human subject will benefit from taking a urolithin supplement, comprising the steps of:
a) Measuring the level of a urolithin or a urolithin conjugate in a first dried blood spot sample, collected prior to administration of a test dose of urolithin;
b) Administering a test dose of a urolithin;
c) Measuring the level of the urolithin or a urolithin conjugate in a second dried blood spot sample, collected after a time interval, for example, about 6-8 hours or about 24 hours; and
d) Determining whether the subject is one who will benefit from ingesting a urolithin supplement from the increase in level of urolithin or urolithin conjugate in the dried blood spot sample; and
e) presenting on a user interface the treatment dose of urolithin supplement for the subject.

In a further embodiment of the invention, there is provided a system for presenting whether a human subject will benefit from taking a urolithin supplement, comprising the steps of:
a) Measuring the level of a urolithin or a urolithin conjugate in a first dried blood spot sample, collected prior to administration of a urolithin precursor, for example, pomegranate juice;
b) Administering the urolithin precursor;
c) Measuring the level of the urolithin or a urolithin conjugate in a second dried blood spot sample; and
d) Determining whether the subject is one who will benefit from ingesting a urolithin supplement from the increase in the level of urolithin or urolithin conjugate in the second dried blood spot sample compared to the first dried blood spot sample; and
e) presenting on a user interface the treatment dose of urolithin supplement for the subject.

In a further embodiment of the invention, there is provided a system for presenting whether a human subject will benefit from taking a urolithin supplement, comprising the steps of:
a) Measuring the level of a urolithin or a urolithin conjugate in a first urine sample, collected prior to administration of a test dose of urolithin;
b) Administering a test dose of a urolithin;
c) Measuring the level of the urolithin or a urolithin conjugate in a second urine sample; and
d) Determining whether the subject is one who will benefit from ingesting a urolithin supplement from the increase in level of urolithin or urolithin conjugate in the urine sample; and
e) presenting on a user interface the treatment dose of urolithin supplement for the subject.

In a further embodiment of the invention, there is provided a system for presenting whether a human subject will benefit from taking a urolithin supplement, comprising the steps of:
a) Measuring the level of a urolithin or a urolithin conjugate in a first urine sample, collected prior to administration of a urolithin precursor, for example, pomegranate juice;
b) Administering the urolithin precursor;
c) Measuring the level of the urolithin or a urolithin conjugate in a second urine sample; and
d) Determining whether the subject is one who will benefit from ingesting a urolithin supplement from the increase in the level of urolithin or urolithin conjugate in the second urine sample compared to the first urine sample; and
e) presenting on a user interface the benefits of taking a urolithin.

Levels of urolithin or urolithin for use with systems of the invention are as disclosed for methods of the invention as recited above.

### Computer Implemented Methods of the Invention

Methods of the invention may be implemented to aid in sample collecting and results transmission to a subject, using computer implementation. For example, a subject may be provided with a computer program, such as an ‘app’ on a mobile computer device, for example, a mobile phone or tablet computer.

Such a computer program may allow a subject to input data such as:
(i) Record that a biological sample has been collected,
(ii) Aid is sending such a sample for testing, such as facilitating the printing of an address label;
(iii) Optionally inputting data such as time and date, age, sex, weight, ethnicity, sample type etc.

Such data may be shared with a remote computer to aid in sample processing and reporting of results.

Such a computer program, may allow transmission of results to the subject, such as levels of urolithin or urolithin conjugate, whether the subject will benefit from a urolithin supplement and what dose of urolithin supplement is required.

In one embodiment of the invention, there is provided a computer-implemented method comprising the steps of:
(a) Optionally receiving data confirming transit of sample of a biological fluid from a subject;
(b) Receiving data of the level of a urolithin or urolithin conjugate in the biological fluid;
(c) Optionally receiving subject data, for example, time and date, age, sex, weight, ethnicity, sample type etc.
(d) Comparing the level of urolithin or a urolithin conjugate with data correlating the level with a treatment dose of a urolithin, for example, urolithin A, to calculate the treatment dose;
(e) Optionally communicating results to the subject.

In one embodiment of the invention, there is provided: a non-transitory computer-readable medium comprising computer-executable instructions that, when executed by a processor of a computing device, cause the computing device to perform a method, the method comprising
(a) Optionally receiving data confirming transit of sample of a biological fluid from a subject;
(b) Receiving data of the level of a urolithin or urolithin conjugate in the biological fluid;
(c) Optionally receiving subject data, for example, time and date, age, sex, weight, ethnicity, sample type etc.
(d) Comparing the level of urolithin or a urolithin conjugate with data correlating the level with a treatment dose of a urolithin, for example, urolithin A, to calculate the treatment dose;
(e) Optionally communicating results to the subject.

The comparison of the level of a urolithin or urolithin conjugate to determine whether a subject could benefit from a urolithin supplement is as defined above for methods of the invention.

The comparison of the level of urolithin or urolithin conjugate with data correlating the level of urolithin or urolithin conjugate with a treatment dose of a urolithin supplement is as described above for methods of the invention.

A computer device comprising a processor, memory and display, the computing device being arranged to implement a computer-implemented method of the invention or computer executed instructions of the invention. Such computer devices include mobile phones, tablet computers, laptop computers etc.

The results comprise information derived from the level of urolithin or urolithin conjugate in the biological fluid, for example, whether the subject could benefit from the administration of a urolithin supplement and/or the treatment dose of a urolithin supplement.

### Medical Treatments

The invention further provides urolithin compositions for the use in methods of medical treatment. Therefore, according to a further embodiment of the invention there is provided a urolithin supplement for use in a method of treating a human subject wherein the subject is one who has been determined as benefiting from taking a urolithin supplement by a method of the invention.

According to a further embodiment of the invention, there is provided a method of treatment comprising determining whether the subject will benefit from taking a urolithin supplement according to a method of the invention and then administering a urolithin supplement to the subject.

According to a further embodiment of the invention there is provided the use of a urolithin supplement in the manufacture of a medicament for treating a disease, disorder of condition in a human subject wherein the subject is one who has been determined as benefiting from taking a urolithin supplement by a method of the invention.

According to a further embodiment of the invention there is provided a method of treating a human subject with a urolithin comprising determining the treatment dose of a urolithin supplement according to a method of the invention and then administering a urolithin supplement to the subject.

According to a further embodiment of the invention, there is provided a method of treatment comprising determining the treatment dose of a urolithin supplement according to a method of the invention and then administering a urolithin supplement to the subject.

According to a further embodiment of the invention there is provided the use of a urolithin supplement in the manufacture of a medicament for treating a disease, disorder of condition in a human subject wherein the treatment dose has been determined by a method of the invention.

In a further embodiment, there is provided use of a calibrated dose of a urolithin for treating a disease, disorder of condition in a human subject.

In a further embodiment, there is proved the use of a calibrated dose of a urolithin in the manufacture of a medicament for treating a disease, disorder of condition in a human subject.

In a further embodiment there is provided a method of treating a disease, disorder of condition in a human subject using a calibrated dose of a urolithin.

A calibrated dose is one which has been determined using a method of the invention.

In a further embodiment of the invention there is provide a urolithin supplement for use in a method of treating a human subject comprising determining a treatment dose of a urolithin supplement according to a method of the invention.

In a further embodiment of the invention, there is provided a urolithin for use in a method of preventing or treating symptoms of aging in a human subject wherein the method comprises administering a urolithin supplement to the subject and wherein the subject is one who has been determined as benefiting from taking a urolithin supplement by a method of the invention or a subject for whom a treatment dose have been calculated using a method of the invention.

The invention provides a method of treating a muscle-related pathological condition in a subject according to a method of the invention. Muscle-related pathological conditions include both conditions impacting generally healthy individuals as well as pathological conditions. Such muscle conditions found in healthy people or people affected by a disease include musculoskeletal diseases or disorders; cachexia; muscle wasting; myopathies; age-related decline in muscle function; pre-frailty; frailty; neuromuscular diseases, such as Duchenne muscular dystrophy and other dystrophies; sarcopenia, for example, acute sarcopenia; muscle atrophy and/or cachexia, for example muscle atrophy and/or cachexia associated with burns, bed rest, limb immobilization, or major thoracic, abdominal, and/or orthopedic surgery; multiple sclerosis, for example relapse remitting form thereof; and muscle degenerative disease.

Examples of age-related conditions that may be treated with compositions of the invention include sarcopenia, pre-frailty, frailty, swallowing difficulties or dysphagia, and muscle wasting. Generally, the treatments improve mitochondrial function associated with age-related decline in muscle function and/or mobility.

In a further embodiment, the invention provides methods for the treatment of a disease or condition selected from the group consisting of metabolic syndrome, reduced metabolic rate, metabolic stress, cardiovascular disease, sarcopenia, muscle degenerative disease, inclusion body myositis (for example sporadic inclusion body myositis), Duchenne muscular dystrophy, alcoholic liver disease, non-alcoholic fatty liver disease (NAFLD), Non-alcoholic steatohepatitis (NASH), drug-induced liver injury, drug-induced cravings, anaemia disorders, al antitrypsin deficiency, ischemia/reperfusion injury, inflammation, inflammatory bowel disease, Crohn's disease, obesity, metabolic syndrome, type II diabetes mellitus, hyperlipidaemia, osteoarthritis, neurodegenerative disease, Alzheimer's disease, Parkinson's disease, Huntington's disease, anxiety disorder, ulceration, amyotrophic lateral sclerosis, mitochondrial diseases (including for example poor growth, loss of muscle coordination, muscle weakness, visual problems, hearing problems, heart disease, liver disease, kidney disease, gastrointestinal disorders, respiratory disorders, neurological problems, autonomic dysfunction sometimes learning disabilities, and dementia as a result of mitochondrial disease. Further diseases related to mitochondrial dysfunction include: Diabetes mellitus and deafness (DAD); Leber's hereditary optic neuropathy (LHON); Leigh syndrome (subacute sclerosing encephalopathy); neuropathy, ataxia, retinitis pigmentosa, and ptosis (NARP); myoneurogenic gastrointestinal encephalopathy (MNGIE); Myoclonic Epilepsy with Ragged Red Fibres (MERRF); Mitochondrial myopathy, encephalomyopathy, lactic acidosis, stroke-like symptoms (MELAS); and mtDNA depletion) and cancer, cognitive disorder, stress, and mood disorder; for improving cognitive function; for weight management; or to increase muscle or mental performance. The compositions of the invention are particularly suitable for use in improving muscle function, muscle strength endurance and muscle recovery.

In particular, the invention provides the treatment of a disease or condition selected from the group consisting of metabolic syndrome, reduced metabolic rate, metabolic stress, cardiovascular disease, sarcopenia, pre-frailty, frailty, muscle degenerative disease, inclusion body myositis (for example sporadic inclusion body myositis), Duchenne muscular dystrophy, alcoholic liver disease, non-alcoholic fatty liver disease, drug-induced liver injury, drug-induced cravings, anaemia disorders, α1 antitrypsin deficiency, ischemia/reperfusion injury, inflammation, inflammatory bowel disease, Crohn's disease, obesity, metabolic syndrome, type II diabetes mellitus, hyperlipidaemia, osteoarthritis, neurodegenerative disease, Alzheimer's disease, Parkinson's disease, anxiety disorder, ulceration, amyotrophic lateral sclerosis, and cancer, cognitive disorder, stress, and mood disorder; for improving cognitive function; for weight management; or to increase muscle or mental performance.

The invention further provides the treatment of a disease or condition selected from the group consisting of metabolic stress, sarcopenia, muscle degenerative disease, inclusion body myositis (for example sporadic inclusion body myositis), Duchenne muscular dystrophy, alcoholic liver disease, non-alcoholic fatty liver disease, drug-induced liver injury, al-antitrypsin deficiency, ischemia/reperfusion injury, inflammatory bowel disease, Crohn's disease, Alzheimer's disease, Parkinson's disease, amyotrophic lateral sclerosis, and cancer.

The invention further provides methods for increasing autophagy or mitophagy in a cell comprising a treatment method of the invention. For example, the autophagy or mitophagy may be in embryonic stem cells, induced pluripotent stem cells, adult stem cells, differentiated cells, blood cells, hematopoietic cells, epithelial cells, exocrine cells, endocrine cells, connective tissue cells, adipose cells, bone cells, smooth muscle cells, striated muscle cells, nerve cells, sensory cells, cardiac cells, hepatic cells, gastric cells, intestinal cells, pulmonary cells, epidermal (i.e. skin) cells (including keratinocytes and fibroblasts), kidney cells, and germ cells. It may thus for example treat or prevent a disease or condition selected from the group consisting of metabolic syndrome, reduced metabolic rate, metabolic stress, cardiovascular disease, sarcopenia, muscle degenerative disease, inclusion body myositis (for example sporadic inclusion body myositis), Duchenne muscular dystrophy, alcoholic liver disease, non-alcoholic fatty liver disease, drug-induced liver injury, drug-induced cravings, anaemia disorders, α1-antitrypsin deficiency, ischemia/reperfusion injury, inflammation, inflammatory bowel disease, Crohn's disease, obesity, metabolic syndrome, type II diabetes mellitus, hyperlipidaemia, osteoarthritis, neurodegenerative disease, Alzheimer's disease, Parkinson's disease, anxiety disorder, ulceration, amyotrophic lateral sclerosis, and cancer, cognitive disorder, stress, and mood disorder; or it can assist with weight management, or increase muscle or mental performance.

Amongst the neurodegenerative diseases, there may specifically be mentioned AIDS dementia complex, Alzheimer's disease, amyotrophic lateral sclerosis, adrenoleukodystrophy, Alexander disease, Alper's disease, ataxia telangiectasia, Batten disease, bovine spongiform encephalopathy (BSE), Canavan disease, corticobasal degeneration, Creutzfeldt-Jakob disease, dementia with Lewy bodies, fatal familial insomnia, frontotemporal lobar degeneration, Huntington's disease, Kennedy's disease, Krabbe disease, Lyme disease, Machado-Joseph disease, multiple sclerosis, multiple system atrophy, neuroacanthocytosis, Niemann-Pick disease, Parkinson's disease, Pick's disease, primary lateral sclerosis, progressive supranuclear palsy, Refsum disease, Sandhoff disease, diffuse myelinoclastic sclerosis, spinocerebellar ataxia, subacute combined degeneration of spinal cord, tabes dorsalis, Tay-Sachs disease, toxic encephalopathy, transmissible spongiform encephalopathy, and wobbly hedgehog syndrome. In one embodiment, the neurodegenerative disease is selected from the group consisting of Alzheimer's disease, amyotrophic lateral sclerosis, Huntington's disease, and Parkinson's disease. In one embodiment, the neurodegenerative disease is Alzheimer's disease.

An aspect of the invention is in improving cognitive function. In one embodiment, the cognitive function is selected from the group consisting of perception, memory, attention, speech comprehension, speech generation, reading comprehension, creation of imagery, learning, and reasoning. In one embodiment, the cognitive function is selected from the group consisting of perception, memory, attention, and reasoning. In one embodiment, the cognitive function is memory.

An aspect of the invention is in the treatment of stress-induced or stress-related cognitive deficit. An aspect of the invention is in the treatment of a mood disorder. In one embodiment, the mood disorder is selected from the group consisting of depression, postpartum depression, dysthymia, and bipolar disorder. In one embodiment, the mood disorder is depression. In one embodiment, the mood disorder is dysthymia.

An aspect of the invention is in the treatment of stress-induced or stress-related mood disorder, e.g., dysthymia. An aspect of the invention is in the treatment of an anxiety disorder. In one embodiment, the anxiety disorder is selected from the group consisting of generalized anxiety disorder, panic disorder, panic disorder with agoraphobia, agoraphobia, social anxiety disorder, obsessive-compulsive disorder, and post-traumatic stress disorder. In one embodiment, the anxiety disorder is generalized anxiety disorder. In one embodiment, the anxiety disorder is post-traumatic stress disorder.

An aspect of the invention is in the treatment of stress-induced or stress-related anxiety.

An aspect of the invention is in the treatment of a muscle or neuromuscular disease. In one embodiment, the muscle or neuromuscular disease is a myopathy. In one embodiment, the muscle or neuromuscular disease is sarcopenia. In one embodiment, the muscle or neuromuscular disease is a muscular dystrophy. In one embodiment, the muscle or neuromuscular disease is Duchenne muscular dystrophy. In one embodiment, the muscle or neuromuscular disease is inclusion body myositis, for example sporadic inclusion body myositis.

An aspect of the invention is in the treatment of mitochondrial disease. For example, a subject may require treatment of loss of muscle coordination, muscle weakness, visual problems, hearing problems, heart disease, liver disease, kidney disease, gastrointestinal disorders, respiratory disorders, neurological problems, autonomic dysfunction sometimes learning disabilities, and dementia as a result of mitochondrial disease.

An aspect of the invention is in enhancing muscle performance. In one embodiment, the muscle performance is selected from the group consisting of strength, speed, endurance and recovery. In humans, muscle function generally declines with age starting during the third decade of life; the decline generally accelerates after age 65. An aspect of the invention is thus in maintaining muscle performance during the aging process. The enhancement of muscle performance may be as part of the use of the compounds in sports nutrition, in aiding healthy aging (for example from age 45 to 65), and in slowing the rate of muscle decline in those aged over 65 (pre-frail)

### Non-Medical Treatments:

The compositions of the invention find use in improving muscle performance, improving muscle function, preventing a decline in muscle function, increasing muscle mass and/or reducing muscle wasting. The improvement in muscle performance, improving muscle function, the increase in muscle mass and/or reduction in muscle wasting may be as part of a medical treatment, or it may be for personal preference (“lifestyle”) or cosmetic reasons, or as part of personal non-prescribed management of nutritional or physiological wellbeing. The compositions of the invention can be for use as a medicament. The compositions can be used as a dietary supplement, as a functional food, functional beverage, specialised nutrition or as a medical food.

The invention provides the treatment of both diseases and disease states. The compositions find use in the management normal physiological function in healthy individuals of conditions characterised by poor physical performance, impaired endurance capacity, and impaired muscle function. Treatment methods of the invention may improve physical performance in individuals with a disease, including young and elderly individuals. Treatment methods of the invention may improve physical performance, for example, short-term performance or long-term performance in healthy individuals, including athletes, non-athletic individuals, sedentary individuals and the elderly. This improvement of performance may be measured by the time spent to walk or run a certain distance (for example, an improved performance during the 6 minute walk test (MWT)), an improved time to run a certain distance, an improved IPAQ score on the international physical activity questionnaire, an increased number of chair-stands in a certain time, or another test designed to measure physical performance.

Treatment methods of the invention also find use in the management and maintenance of normal physiological function (for example physical performance, endurance capacity and muscle function) in healthy individuals.

Treatment methods of the invention also find use in managing a nutritional state that leads to improved mitochondrial function. This is important, for example, in people having a disease or hospitalized, where the urolithin supplement is administered not to treat a disease but as a nutritional supplement.

Treatment methods of the invention further provide for the improvement of endurance capacity. The endurance capacity refers to the time to fatigue when exercising at a constant workload, generally at an intensity <80% VO2max. Treatment methods of the invention may improve endurance capacity in individuals with a disease, including young and elderly individuals. Treatment methods of the invention may improve endurance capacity in healthy individuals, including athletes, non-athletic individuals, sedentary individuals and the elderly. The invention provides for a method of increasing the time to fatigue while performing a specific activity, for example, fitness training, walking, running, swimming, or cycling. This improvement of endurance capacity may be assessed with objective measurements (for example, speed, oxygen consumption or heart rate) or it can be self-reported measurements (for example, using a validated questionnaire).

The invention further provides a treatment methods to improve, maintain or reduce the loss of muscle function. Compositions of the invention may improve, maintain or reduce the loss of muscle function in individuals with a disease, including young and elderly individuals. Compositions of the invention may improve, maintain or reduce the loss of muscle function in healthy individuals, including athletes, non-athletic individuals, sedentary individuals and the elderly. For example, compositions of the invention may improve, maintain or reduce the loss of muscle function in frail or pre-frail individuals. For example, compositions of the invention may increase muscle strength as evidenced by the improvement of performing a physical activity, such as an exercise, for example, increased ability to lift weights or increased hand grip strength. Also, compositions of the invention may improve muscle structure, for example by increasing or maintaining muscle mass in conditions of normal muscle function, declining muscle function or impaired muscle function.

This invention further provides treatment methods to improve the physical performance or endurance capacity as perceived by the individual. For example, by the reduction of in perceived exertion or effort during exercise or an activity as determined using a self-reported questionnaire.

In a further embodiment of the invention there is provided a method of maintaining adequate nutrient levels in a human subject wherein the method comprises administering a urolithin supplement to the subject and wherein the subject is one who has been determined as benefiting from taking a urolithin supplement or determining a dose of a urolithin supplement as described herein.

### Muscle Performance:

The treatment methods of the invention are useful in enhancing muscle and/or physical performance. The invention thus provides a composition of the invention for use in enhancing muscle and/or physical performance. The invention also provides a method of enhancing muscle and/or physical performance by administering to a subject an effective amount of a composition of the invention. Administration can be self-administration.

The enhanced muscle performance may be one or more improved muscle function, reduced decline in muscle function, maintenance of muscle function, improved muscle strength, improved or maintenance of muscle endurance and improved muscle recovery.

The treatment methods of the invention can thus be used in a method of improving physical endurance (e.g., ability to perform a physical task such as exercise, physical labour, sports activities), inhibiting or retarding physical fatigue, enhancing working capacity and endurance, and reducing muscle fatigue.

Improved muscle function can be particularly beneficial in elderly subjects with reduced muscle function as a result of an age-related condition, for example sarcopenia and muscle wasting. The treatment methods of the invention may be used in enhancing muscle performance by administering a composition of the invention to a subject who is sedentary, frail or pre-frail.

Muscle performance may be sports performance, which is to say the ability of an athlete's muscles to perform when participating in sports activities. Enhanced sports performance, strength, speed, and endurance are measured by an increase in muscular contraction strength, increase in amplitude of muscle contraction, or shortening of muscle reaction time between stimulation and contraction. Athlete refers to an individual who participates in sports at any level and who seeks to achieve an improved level of strength, speed, or endurance in their performance, such as, for example, body builders, bicyclists, long distance runners, and short distance runners. Enhanced sports performance is manifested by the ability to overcome muscle fatigue, ability to maintain activity for longer periods of time, and have a more effective workout.

### Urolithins

Urolithins are metabolites produced by the action of mammalian, including human, gut microbiota on ellagitannins and ellagic acid. Ellagitannins and ellagic acid are compounds commonly found in foods such as pomegranates, nuts and berries. Ellagitannins are minimally absorbed in the gut themselves. Urolithins are a class of compounds with the representative structure (I) shown above. The structures of some particularly common urolithins are described in Table 1 below, with reference to structure (I).

| | |
|---|---|
| | (I) |
| 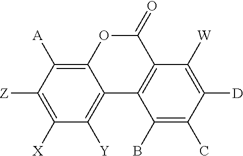 | |

| | Substituent of structure (I) | | | | | |
|---|---|---|---|---|---|---|
| | A | B | C | D | W, X and Y | Z |
| Urolithin A | H | H | H | OH | H | OH |
| Urolithin B | H | H | H | H | H | OH |
| Urolithin C | H | H | OH | OH | H | OH |
| Urolithin D | OH | H | OH | OH | H | OH |
| Urolithin E | OH | OH | H | OH | H | OH |
| Isourolithin A | H | H | OH | H | H | OH |
| Isourolithin B | H | H | OH | H | H | H |
| Urolithin M-5 | OH | OH | OH | OH | H | OH |
| Urolithin M-6 | H | OH | OH | OH | H | OH |
| Urolithin M-7 | H | OH | H | OH | H | OH |

In practice, for commercial scale products, it is convenient to synthesise the urolithins. Routes of synthesis are described, for example, in WO 2014/004902 and WO 2015/100213.

Urolithins of any structure according to structure (I) may be used in the methods of the present disclosure.

In one aspect of the uses and methods of the present disclosure, a suitable compound is a compound of formula (I) wherein A, C, D and Z are independently selected from H and OH and B, W, X and Y are all H, preferably at least one of A, C, D and Z is OH.

Particularly suitable compounds are the naturally-occurring urolithins. Thus, Z is preferably OH and W, X and Y are preferably all H. When W, X and Y are all H, and A, and B are both H, and C, D and Z are all OH, then the compound is Urolithin C. When W, X and Y are all H, and A, B and C are all H, and D and Z are both OH, then the compound is urolithin A. Preferably, the urolithin used in the methods of the present disclosure is urolithin A, urolithin B, urolithin C or urolithin D. Most preferably, the urolithin used is urolithin A.

According to one embodiment there is provided a method of the invention wherein the compound of formula (I) is urolithin A.

According to one embodiment there is provided a method of the invention wherein the compound of formula (I) is urolithin B.

According to one embodiment there is provided a method of the invention wherein the compound of formula (I) is urolithin C.

According to one embodiment there is provided a method of the invention wherein the compound of formula (I) is urolithin D.

Forms of Compositions:

The compositions for use in treatment methods of the invention can take any suitable physical form. They may be in the form of a solid (for example a tablet or a bar), a semi-solid (for example a softgel, capsule (for example a hard capsule) or dragee), a powder or a liquid (including emulsions). The compositions of the invention may be nutritional compositions. The compositions of the invention may be pharmaceutical compositions. The compositions can be in the form of a dietary supplement, as a functional food, functional beverage, or as a medical food or medical nutrition product.

Tablet form compositions may be of any suitable type, and they may contain excipients conventional in the art. The excipients can, for example, provide a desired hardness, shelf-life and flavour such that the compostions has an acceptable taste, an attractive appearance and good storage stability. A bar may be of any suitable type and it may contain ingredients conventionally used for the preparation of snack bars.

Semi-solid forms may likewise contain excipients conventional in the art. The excipients can, for example, provide a desired hardness, shelf-life and flavour such that the composition has an acceptable taste, an attractive appearance and good storage stability. Semi-solid forms may be provided for oral administration, or for topical administration.

Powders are commonly used for the supply of nutritional and medical compositions. Powders have the advantage that multiple doses can be provided in a single container, and doses of various sizes can be used from the same supplied container. Powders generally have good storage properties. Powder compositions may also contain excipients conventional in the art. The excipients can, for example, provide a shelf-life, flavour and moisture resistance such that the composition has an acceptable taste, an attractive appearance and good storage stability. The current invention may take the form of a kit comprising a nicotinamide riboside composition together with a separate composition containing urolithin, for example nicotinamide riboside powder composition together with a separate solid or liquid composition containing urolithin. A solid or liquid composition containing urolithin (for example a tablet or a drink, or other form described herein) may be provided with instructions for use together with a nicotinamide riboside powder. For example, both nicotinamide riboside and urolithin may be in powdered form.

Liquid compositions may be in the form of a medicine, in the form of a drink. Liquid formulations may be solutions, emulsions, slurries or other semi-liquids. Excipients in a liquid composition can, for example, provide a shelf-life, visual appearance, flavour and mouthfeel such that the composition has an acceptable taste, an attractive appearance and good storage stability. Liquid compositions may be provided for oral administration. Liquid compositions may be provided for topical application, for example in the form of creams, ointments or lotions.

For some uses, compositions of the invention may also be in the form of a solution suitable for injection or intravenous administration.

Additional Components in Compositions of the Invention:

The compositions for use in treatment methods of the invention may contain additional components beyond the urolithin and the nicotinamide riboside. The additional components may be compounds that provide health benefits, for example selected from vitamins, minerals, polyunsaturated fatty acids, functional amino acids and other compounds.

Amongst vitamins, there may specifically be mentioned Vitamin A, Vitamin C, Vitamin D, Vitamin E, Vitamin B12 and Vitamin K2. As used herein, “vitamin D” refers, to any of known form of vitamin D, and specifically includes vitamin D2 (ergocalciferol), vitamin D3 (cholecalciferol), vitamin D precursors, metabolites and other analogues, and combinations thereof, as well as the various active and inactive forms of vitamin D. For example, vitamin D3 may be provided in its unhydroxylated inactive form as cholecalciferol, or may be provided in its hydroxylated active form as calcitriol.

Creatine has been described as having beneficial effects in the treatment of muscle disorders. It can be included in compositions of the invention. β-hydroxyl-β-methylbutyrate (HMB) has been described as having beneficial effects in the treatment of muscle disorders. It can be included in compositions of the invention.

Amongst minerals, there may specifically be mentioned calcium salts (for example calcium phosphate), selenium, zinc salt, magnesium salts and iron salts.

For many muscle growth and/or muscle enhancement treatments, it is beneficial for certain particular amino acids to be provided. For example, L-arginine, L-glutamine, lysine and the branched-chain amino acids are considered important. These amino acids are sometimes known as “functional amino acids”. The composition of the invention may include one or more branched-chain amino acids (leucine, isoleucine, and valine). The composition of the invention may include one or both of L-arginine and L-glutamine. The composition of the invention may include lysine.

Pharmaceutical compositions of the invention may include additional pharmaceutically active compounds. For example, a statin may be included. The invention may be provided as a kit comprising a composition of urolithin and nicotinamide riboside; and a pharmaceutically active compound, for example a statin.

A composition of the invention may include one or more further agents that are useful for mitochondrial biogenesis or the treatment of mitochondrial disorders. Such compounds include, without limitation, resveratrol, pyrroloquinoline quinone, ubiquinone, sulforaphane, co-enzyme Q10, genistein, hydroxyltyrosol, quercetin, L-carnitine, alpha-lipoic acid, and folinic acid (e.g., as leucovorin).

Additional compounds may further (or alternatively) be included in a composition of the invention, including for example tomatidine, ursolic acid, curcumin, capsaicin, menthol, trolamine salicylate and methylsalicylate.

In some exemplary embodiments, the compositions of the present disclosure may comprise, in addition to nicotinamide riboside and urolithin, one or more additional macronutrients, typically protein, fat or carbohydrate, or two or more of protein, fat and carbohydrate.

Any suitable source of fat or oil of the type commonly used in the preparation of foodstuffs and pharmaceuticals may be used in compositions of the invention. Non-limiting examples of suitable sources of fats for use in the compositions described herein also include polyunsaturated fatty acids such as docosahexaenoic acid (DHA), arachidonic acid (ARA), eicosapentaenoic acid (EPA) and combinations thereof.

Non-limiting examples of suitable carbohydrates or sources thereof for use in the compositions described herein may include maltodextrin, hydrolyzed or modified starch or cornstarch, glucose polymers, corn syrup, corn syrup solids, rice-derived carbohydrates, glucose, fructose, lactose, trehalose, high fructose corn syrup, tapioca dextrin, isomaltulose, sucromalt, maltitol powder, glycerin, fructooligosaccharides, soy fiber, corn fiber, guar gum, konjac flour, polydextrose, honey, sugar alcohols (e.g., maltitol, erythritol, sorbitol), and combinations thereof. Maltodextrin, sucrose and fructose are especially preferred.

Non-limiting examples of suitable proteins or sources thereof for use in the compositions described herein may include hydrolyzed, partially hydrolyzed or non-hydrolyzed proteins or protein sources. They may be derived from any known or otherwise suitable source such as milk (e.g., casein, whey), animal (e.g., meat, fish), cereal (e.g., rice, corn) or vegetable (e.g., soy, pea) sources. Combinations of sources or types of proteins may be used. Non-limiting examples of proteins or sources thereof include intact pea protein, intact pea protein isolates, intact pea protein concentrates, milk protein isolates, milk protein concentrates, casein protein isolates, casein protein concentrates, whey protein concentrates, whey protein isolates, sodium or calcium casemates, whole cow's milk, partially or completely defatted milk, yoghurt, soy protein isolates and soy protein concentrates, and combinations thereof. Combinations of sources or types of proteins may be used. For example, Greek- and Icelandic-style yoghurts are known to commonly have an especially high protein content which makes them especially suitable for use in formulations of the invention. Yoghurts for use in compositions of the invention may contain, for example, from 2 to 15 g of protein per 100 g. Particularly preferred are yoghurts with a high protein content, for example from 6 to 15 g per 100 g, for example from 7 to 15 g per 100 g, for example from 8 to 15 g per 100 g. Optionally, supplemental protein may also be added to a yoghurt formulation to increase the protein content of the formulation. Yoghurts of the invention may contain live cultures, such as S. thermophilus, L. bulgaricus, L. acidophilus or L. lactis.

The total concentrations or amounts of the protein, fat, carbohydrates and other components vary depending upon the nutritional needs of the intended user.

Additional components in a composition of the invention may be compounds that do not provide health benefits to the subject, but instead improve the composition in some other way, for example its taste, texture or shelf-life as mentioned above. The composition of the invention may thus further contain one or more compounds selected from emulsifiers, colorants, preservatives, gums, setting agents, thickeners, sweeteners and flavourings.

Suitable emulsifiers, colorants, preservatives, gums, setting agents and thickeners are well known in the art of manufacture of emulsions and other semi-liquids. For example preservatives, such as benzoic acid, sorbic acid, phosphoric acid, lactic acid, acetic acid, hydrochloric acid and the soluble salts thereof may be used.

A sweetener may be especially beneficial in a composition of the invention. High potency non-nutritive carbohydrate sweetening agents may be used, for example selected from aspartame, sucrose, potassium acelsufame, saccharin, cyclamates, Stevia, thaumatin and mixtures thereof. Aspartame is especially suitable.

A flavouring may be especially beneficial in a composition of the invention. In a liquid or semi-liquid composition, fruit flavour can be provided by inclusion of a fruit sauce or puree. Typical flavorings include strawberry, raspberry, blueberry, apricot, pomegranate, peach, pineapple, lemon, orange and apple. Generally, fruit flavourings include fruit extract, fruit preserve or fruit puree, with any of a combination of sweeteners, starch, stabilizer, natural and/or artificial flavours, colourings, preservatives, water and citric acid or other suitable acid to control the pH.

For oral preparations, the compositions can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavouring agents.

The compositions can be formulated into liquid preparations by dissolving, suspending or emulsifying them in an aqueous or nonaqueous solvent, such as vegetable or other similar oils, synthetic aliphatic acid glycerides, esters of higher aliphatic acids or propylene glycol; and if desired with conventional additives such as solubilizers, isotonic agents, suspending agents, emulsifying agents, stabilizers and preservatives. The compositions can be utilized in aerosol formulation to be administered via inhalation. They can be made into suppositories by mixing with a variety of bases such as emulsifying bases or water-soluble bases.

Unit dosage forms for oral administration such as syrups, elixirs, and suspensions may be provided wherein each dosage unit, for example, teaspoonful, tablespoonful, tablet or capsule, contains a predetermined amount of a composition of the invention. Similarly, unit dosage forms for injection or intravenous administration may comprise the compound of the present invention in a composition as a solution in sterile water, normal saline or another pharmaceutically acceptable carrier, wherein each dosage unit, for example, mL or L, contains a predetermined amount of a composition of the invention.

The composition for use in treatment methods of the invention can be taken as a single treatment or, more commonly, as a series of treatments. In one example, a subject takes a dose before or after exercise. For a subject who is not able to exercise, a dose of the composition may, for example, be taken once, twice or three times per day, or one, two, three, four, five or six times per week. It will also be appreciated that the effective dosage of the compound may increase or decrease over the course of a particular treatment.

### Dosing

The dose suitable of a urolithin supplement for treatment of the human subject will depend on their degree of formation of urolithin, as indicated by the results of the diagnostic assays of the invention and the optimum dose for a therapeutic benefit in the patient. That is the most suitable dose will be optimum dose minus the amount of urolithin produced by the subject. For example, a dose in the range of 50 mg per day to 2000 mg per day. For example, a dose of 50 mg per day to 1500 mg per day. For example, 50 mg per day to 1400 mg per day, such as to 1300 mg per day, to 1200 mg per day, to 1100 mg per day, such as 50 mg to 1000 mg per day. In a further embodiment the suitable dosage form may be form 100 mg per day to 1000 mg per day, such as 100 mg per day to 750 mg per day, such as 100 mg to 500 mg per day, such as 250 mg to 500 mg per day. For example, the dose of urolithin supplement is selected from 100 mg per day, 250 mg per day, 500 mg per day or 1000 mg per day or 1500 mg/day, such as 250 mg per day, 500 mg per day or 1000 mg per day or 1500 mg/day.

In a further embodiment, a suitable dose of a urolithin supplement may be a unit dose comprising a urolithin or a multiple of a unit dosage form comprising a tablet. For example, a unit doses comprising 100 mg, 250 mg, 500 mg, 1000 mg or 1500 mg. For the avoidance of doubt, doses can be made up of multiples or combinations of the unit doses above.

The suitable dose of a urolithin supplement may also vary depending upon the manner of administration, the age, body weight, and general health of the subject. Factors such as the disease state, age, and weight of the subject may be important, and the suitable dose may be adjusted to provide the optimum response.

In the specification, where concentrations of urolithin are expressed as ng/mL for dry blood spots, this relates to the concentration in ng/mL in the original blood sample, prior to drying onto a support such as a piece of filter paper.

The term ‘about’ refers to a tolerance of ±20% of the relevant value, for example ±15% of the relevant value, such as ±10% of the relevant value or ±5% of the relevant value.

The phrase ‘sample of a biological fluid’ refers to any sample derived from a subject, e.g. a human or animal subject, in which a urolithin or urolithin conjugate can be measured. For example, urine or blood. In the case of blood, the blood can be analysed in the form of a dried blood spot.

The term ‘dried blood spot’ refers to a sample of whole blood, plasma or serum which has been dried on a material, wherein the dried sample can be used to measure the level of urolithin or a urolithin conjugate in the sample. For the avoidance of doubt, the term ‘dried drug spot’ refers to any sample of dried whole blood, dried plasma or dried serum, whether absorbed onto a material or dried as a layer on said material or dried whole blood, plasma or serum scrapped off said material

The term ‘human subject’ relates to a man, woman or child. The man or woman may be young, old or middle aged. The term ‘child’ refers to anyone between about 1 year and about 18 years of age. The term ‘young’ refers to anyone between about 18 years of age and 40 years of age. The term ‘middle aged’ refers to anyone between about 40 years of age and 65 years of age. The term ‘old’ refers to anyone above 65 years of age.

The term ‘urolithin conjugate’ refers to any urolithin derivative formed by metabolism of urolithin in a human subject. Examples of urolithin conjugates include urolithin glucuronide and urolithin sulphate, particularly urolithin glucuronide. In one embodiment methods of the invention comprise measuring the level of a urolithin conjugate, particularly urolithin glucuronide.

The term ‘urolithin precursor’ refers to a plant-derived material or substance, which can be metabolised in the human intestine to form a urolithin. Examples of plant-derived materials include: tea, fruits such as pomegranates, guava and; berries such as raspberries, blackberries, cloud berries, Boysenberries, strawberries and cranberries; and nuts, such as pecans, walnuts and almonds and seeds such as cloudberry seeds and beefsteak fungus. Examples of substances include: ellagitannins such as punicalagin and ellagic acid.

### BRIEF DESCRIPTION OF THE FIGURES

FIG. 1: shows the levels of urolithin glucuronide in dried blood spots in subjects pre-treatments and after administration of pomegranate juice and 500 mg urolithin.

FIG. 2: shows a flow chart of the study described in Example 5.

FIG. 3: shows UA-glucuronide levels in plasma samples collected at the timepoints of 0 (baseline), 6 and 24 hrs. post intake after either Pomegranate juice or Urolithin A in all n=100 healthy participants in Example 5. Data shown are Mean of 100 samples.

FIG. 4 shows UA-glucuronide levels in DBS (dried blood spot) samples at the timepoints of 0 (baseline), 6 and 24 hrs. post intake after either Pomegranate juice or Urolithin A in all n=100 healthy participants in Example 5.

FIG. 5A: shows the correlation of DBS (whole blood spot samples) measurements with plasma level measurements for UA glucuronide at t=6 hrs for the n=100 subjects (Spearman correlation coefficient r=0.97).

FIG. 5B: shows the correlation of DBS (whole blood spot samples) measurements with plasma level measurements for UA glucuronide at t=24 hrs for the n=100 subjects (Spearman correlation coefficient r=0.97).

FIG. 6A shows boxplots showing differences in metagenomics species (MGS) for richness (left panel) and Shannon diversity (right panel) between groups with no-, low-, and high-UA producer status. All groups were compared pairwise by Mann-Whitney U test (N=99). *P≤0.05; **P≤0.01; ***P≤0.001; *****P≤0.00001.

FIG. 6B shows principal coordinate analysis (PCoA) based on Bray-Curtis dissimilarities among samples, calculated based on the MGS abundances. Samples are color coded by the UA producer status. Each sample is connected to its group centroid by a thin segment line. The ellipses cover two standard errors of the mean of the group centroids, i.e. they illustrate the certainty of the group centroid positions. The x- and y-axis labels indicate the microbial variance explained by the first two principal coordinates.

FIG. 6C shows relative abundance (in per cent) of phyla Firmicutes and Bacteroidetes which significantly differed in abundance in producer of UA compared with no-producer. Boxes represent interquartile range (IQR), with the inside horizontal line representing the median. Whiskers represent values within 1.5×IQR of the first and third quartiles.

FIG. 6D shows firmicutes/Bacteroidetes ratio (F/B ratio) for each group shown as median (IQR). **P≤0.01 (Mann-Whitney U test).

FIG. 7: shows Boxplots showing differences in microbiome genes for richness (left panel) and Shannon diversity (right panel) between groups with no-, low-, and high-UA producer status. All groups were compared pairwise by Mann-Whitney U test (N=99). *P≤0.05; **P≤0.01; ***P≤0.001, *****P≤0.00001.

FIG. 8: Relative abundance (in per cent) of four MGS and two phyla which significantly differed in abundance or prevalence in UA low-producer compared with no-producer. The relative abundance is shown as the area of each square, scaled to the maximum within each species.

FIG. 9A shows comparison of physiological parameters and food habits between groups with no-, low-, and high-UA producer status. BMI was significantly higher in no- (p=0.02) and low- (p=0.01) UA producers compared with high producers.

FIG. 9B shows comparison of physiological parameters and food habits between groups with no-, low-, and high-UA producer status. Resting diastolic blood pressure was significantly higher in the no-producer group (p=0.017) than the high-producer group.

FIG. 9C shows comparison of physiological parameters and food habits between groups with no-, low-, and high-UA producer status. Heart rate in subjects who were non-producers compared to the high producers (p=0.07).

FIG. 9D shows comparison of physiological parameters and food habits between groups with no-, low-, and high-UA producer status. Fruits and berry intake were higher in the high UA producer group than both the no-producer group (p=0.07) and the low-producer group (p=0.01) as assessed via a dietary questionnaire. N=100. Data are expressed as mean+/−SEM and analyzed using repeated measure ANOVA. *P≤0.05; **P≤0.01.

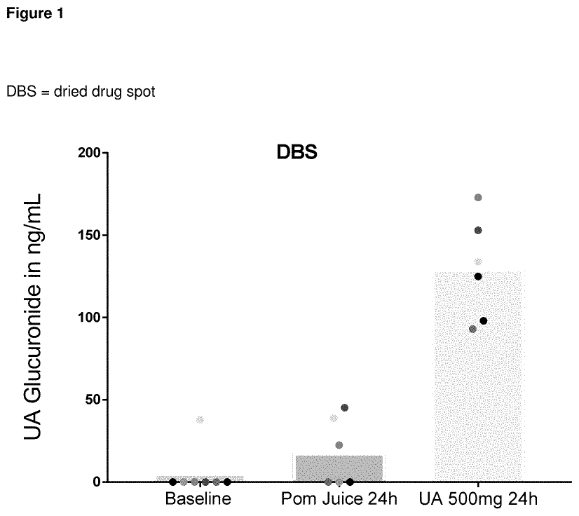

### EXAMPLES

The invention will now be illustrated with respect to the following non-limiting Examples

### Example 1: Preparation of Urolithin A

Urolithin A (4) was prepared in two steps starting from 2-bromo-5-methoxybenzoic acid 1 and resorcinol 2. The pure compound was obtained as a pale yellow powder.

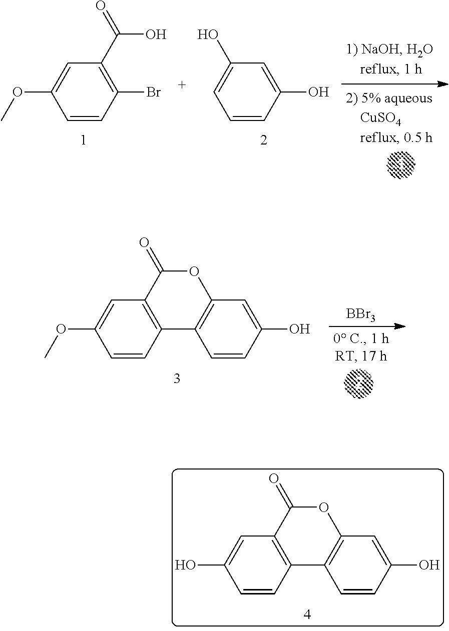 [SMILES: COc1ccc(Br)c(C(=O)O)c1 ; Oc1cccc(O)c1 ; COc1ccc2c(c1)c(=O)oc1cc(O)ccc12 ; "BBr3" ; O=c1oc2cc(O)ccc2c2ccc(O)cc12]

### Step 1:

A mixture of 2-bromo-5-methoxybenzoic acid 1 (27.6 g; 119 mmol; 1.0 eq.), resorcinol 2 (26.3 g; 239 mmol; 2.0 eq.) and sodium hydroxide (10.5 g; 263 mmol; 2.2 eq.) in water (120 mL) was heated under reflux for 1 hour. A 5% aqueous solution of copper sulphate (3.88 g of CuSO4.5H2O in 50 mL water; 15.5 mmol; 0.1 eq.) was then added and the mixture was refluxed for additional 30 minutes. The mixture was allowed to cool to room temperature and the solid was filtered on a Büchner filter. The residue was washed with cold water to give a pale red solid which was triturated in hot MeOH. The suspension was left overnight at 4° C. The resultant precipitate was filtered and washed with cold MeOH to yield the title compound 3 as a pale brown solid.

### Step 2:

To a suspension of 3 (10.0 g; 41 mmol; 1.0 eq.) in dry dichloromethane (100 mL) was added dropwise at 0° C. a 1 M solution of boron tribromide in dry dichloromethane (11.93 mL of pure BBr3 in 110 mL of anhydrous dichloromethane; 124 mmol; 3.0 eq.). The mixture was left at 0° C. for 1 hour and was then allowed to warm up to room temperature. The solution was stirred at that temperature for 17 hours. Then ice was added thoroughly to the mixture. The yellow precipitate was filtered and washed with cold water to give a yellow solid which was heated to reflux in acetic acid for 3 hours. The hot solution was filtered quickly and the precipitate was washed with acetic acid, then with diethyl ether to yield the title compound 4 as a yellow solid. 1H and 13C NMR were in accordance with the structure of 4.

### Example 2: Urolithin a Dosage Form

Urolithin A was formulated into a soft gel capsule containing the following components:

Fill

| Ingredients | Amount (mg)/Cap | % Total |
|---|---|---|
| Urolithin A | 250 | 22.73% |
| Lecithin NF (35% Total PC) | 284.25 | 25.84% |
| (Epikuron 135 F IP) - E322 | | |
| Medium Chain Triglycerides | 284.25 | 25.84% |
| (MCT) | | |
| Glycerol Monostearate (40-55) | 11.5 | 1.06% |
| EP, Mono- and Diglycerides | | |
| NF | | |
| Fill Weight | 830 mg | 75.47% |

Shell

| Ingredients | Amount (mg)/Cap | % Total |
|---|---|---|
| Gelatin EP, NF | 165.97 | 15.09% |
| Glycerol - E422 | 80.01 | 7.27% |
| Water | 21.62 | 1.96% |
| Titanium Dioxide EP - E171 | 1.96 | 0.18% |
| DualDustmaster FD&C Blue | 0.234 | 0.021% |
| #1 (Brilliant Blue FCF - E133) | | |
| Sodium Copper Chlorophyllin | 0.196 | 0.018% |
| Powder (min 95%) - E141 | | |
| Shell Weight | 270 mg | 24.539% |
| Total Capsule Weight | 1100 mg | 100% |

### Example 3—Study of the Effect of Pomegranate Juice and Oral Urolithin on Levels of Blood, Collected in Dried Blood Spots

A study was conducted in seven subjects. At the beginning of the study a sample of capillary blood via a finger prick was taken from each subject and 15-20 uL of whole blood was dried onto a dried blood sample (DBS) filter paper sample card (Whatman, Little Chalfont, Buckinghamshire, UK). A minimum of 2 spots per sampling were collected for each time-point. At T0 i.e. baseline sample, subjects ingested 80 ml pomegranate juice (freshly prepared/). At 24 hours following pomegranate juice intake, a further dried blood spot sample was taken and 15-20 uL of whole blood was dried onto a DBS filter paper card. Following this, subjects ingested 500 mg of Urolithin A in a softgel capsule and then at 48 hours (or 24 hours following UA intake), a further dried blood spot sample was taken and 15-20 uL of whole blood was dried onto DBS filter paper sample card.

### Example 3a: Measurement of Urolithin A Glucuronide in Dried Blood Spot Samples

This analytical method was developed to analyse whole blood spot samples collected on dry blood spot sample cards (DBS Filter card) in Example 3.

Each dried blood spot was punched from cards using a 6 mm diameter puncher and transferred into a tube. Afterwards 150 μL of methanol containing the internal standard was added. Subsequently the samples were undergoing sonication for 5 minutes followed by a vortex mixing step for 20 minutes. Then samples were centrifuged for 2 minutes at 50000 g and 8° C. An aliquot of 100 μL of the supernatant was transferred in an autosampler vial for analysis and diluted with 100 μL of water prior to analysis.

The quantification of UA-glucuronide was performed by column separation with reversed-phase chromatography followed by detection with triple-stage quadrupole MS/MS in the selected reaction monitoring mode.

Results are shown in Table 1

TABLE 1

| | Baseline-Free | Pomegranate | 500 mg single UA |
|---|---|---|---|
| | Living | Juice | dosing |
| | Pre UA levels | 24 h UA levels | 24 h UA levels |
| Subject | (ng/mL) | (ng/mL) | (ng/mL) |
| 1 | 0 | | 125 |
| 2 | 37.9 | 38.8 | 134 |
| 3 | 0 | 22.4 | 173 |
| 4 | 0 | 45.2 | 153 |
| 5 | 0 | 0 | |
| 6 | 0.0 | 0.0 | 93.0 |
| 7 | 0.0 | 0.0 | 98.0 |

### Example 4: Bioavailability Study in Healthy Adults

A single-centre, two-period, crossover, randomized, open labelled study was performed to evaluate the Urolithin A producer status in a healthy US based adult population and to establish the superiority of dietary supplementation UA supplementation in establishing higher Urolithin A circulating levels over dietary exposure.

STUDY POPULATION: Healthy males and females aged between 18-80 years old. At least 20% participants will be between 20-40 years; 41-60 years; and 61-80 years. The remaining 40% distributed among any age group.
SAMPLE SIZE: N=100; all participants completed the study

### Investigational Product

I. 237 mL of Pomegranate Juice (PJ)/day of administration (POM Wonderful, Los Angeles, Calif., USA, 90064).
II. 500 mg Urolithin A (UA) in berry flavoured¹ food sachet/day of administration 1* ingredients that include blueberry, raspberry fruit powder along with rice hull and pomegranate natural flavouring
   The urolithin A powder was admixed into a vanilla flavoured commercial yogurt (4 fl. oz) prior to administration to the participants

### Efficacy Parameters

### Primary Endpoint:

Absolute change from T₀ to T+24 hours in the Urolithin A (UA) group as compared to pomegranate juice (PJ) group in UA-glucuronide plasma levels.

### Secondary Endpoints:

Absolute change from T₀ to T+24 hours in the UA group as compared to PJ group in UA-glucuronide blood spot levels.
Absolute change from T₀ to T+6 hours in the UA group as compared to PJ group in UA-glucuronide plasma levels.
Absolute change from T₀ to T+6 hours in the UA group as compared to PJ group in UA-glucuronide blood spot levels.
Change in AUC from T₀ to T+6 and/or T+24 in the UA group as compared to the PJ group in UA glucuronide levels
Absolute change from T₀ to T6 hours and/or to T24 hours and change in AUC in the UA group as compared to PJ group in the following variables:
   UA sulfate plasma levels
   UA aglycone (parent) plasma levels
To assess prevalence of UA producers following consumption of fixed volume of Pomegranate juice in a healthy US population

Note: “UA producer” status is defined as someone who has detectable UA (and its conjugates) levels in plasma/dried blood spots following pomegranate juice intake. “UA producer” status was decided following review of results at T+24.
Differences, if any, between UA producers and non-producers for the following variables at baseline:
   Age and BMI
   Hand grip strength
   Gait speed
   Physical activity and energy levels via:
   International Physical Activity Questionnaire (IPAQ)
   Fatigue VAS Score
   Dietary intake of foods high in ellagitannins and punicalagins i.e. Nuts and berries
Differences, if any, between UA producers and non-producers in:
   Metabolomic analysis of plasma and/or DBS
   Any of the blood biochemistry parameters measured at visit 1: HbA1c, Creatinine, AST, ALT, Total bilirubin, total cholesterol, HDL, LDL and triglycerides)
   Faecal Analysis via microbiome sequencing to include:
      Sample-specific relative abundance and taxonomic identity of all OTUs (operational taxonomical units)
      Sample-specific relative abundance of bacteria from phylum to genus level
      Alpha and beta diversity measures (e.g. Shannon index and UniFrac distances)
      Identification of phylogenetic groups that change significantly in abundance between UA producer's and non-producers
      Statistical analysis of differences in microbiome community composition and diversity measures between UA producer's and non-producers

### Inclusion Criteria:

To be eligible for inclusion, the subject must have fulfilled all of the following criteria:

1. Males and females 18 to 80 years of age;
2. Is in general good health, as determined by the clinical research team;
3. Willingness to consume investigation product, complete questionnaires and to complete all clinic visits;
4. Have given voluntary, written, informed consent to participant in the study;

### Exclusion Criteria:

The presence of any of the following criteria excluded the subject from participating in the study:
1. Women who were pregnant, breastfeeding, or planning to become pregnant during the course of the trial;
2. Alcohol or drug abuse within the last 6 months;
3. Volunteers who planned to donate blood during the study or within 30 days of completing the study;
4. Subject having a known allergy to the test material's active or inactive ingredients;
5. Subjects with unstable medical conditions;
6. Clinically significant abnormal laboratory results at screening;
7. Participation in a clinical research trial within 30 days prior to randomization;
8. Allergy or sensitivity to study ingredients; lactose intolerance
9. Individuals who were cognitively impaired and/or who were unable to give informed consent;
10. Any other condition which in the Investigator's opinion may adversely affect the subject's ability to complete the study or its measures or which may pose significant risk to the subject;
11. Had taken antibiotics within the previous 30 days.

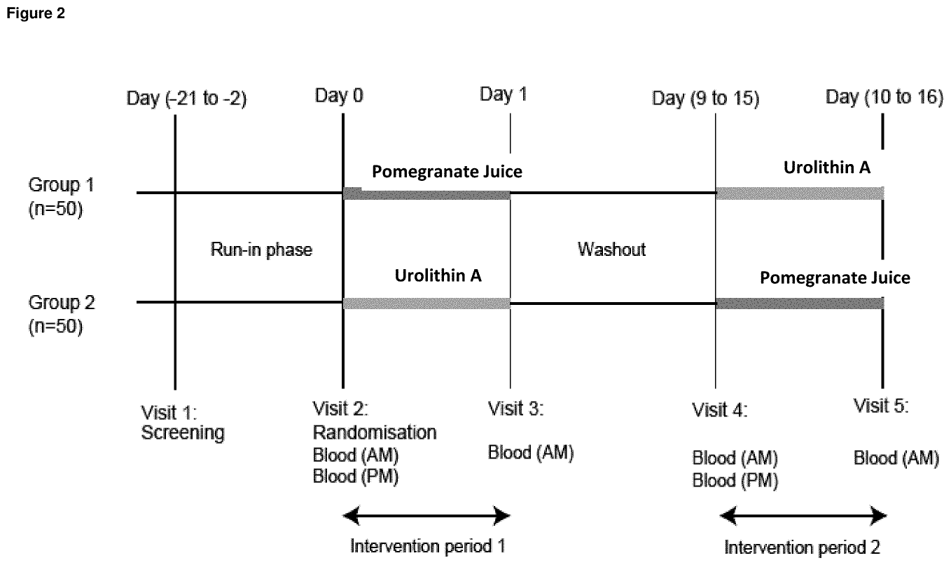

The study flowchart can be seen in FIG. 2.

### Plasma Collection

At each time point indicated in the study assessments table, a 12 mL blood sample was drawn into K2-EDTA coated tube. The blood samples were gently inverted a few times for complete mixing with the anticoagulant. The exact time of sample collection was be recorded on the tube and in the electronic case report form (eCRF). Within 30 minutes following blood collection, each blood sample was centrifuged at 1500 g for 10 minutes at 4° C. Within 30 minutes after the centrifugation, the top layer of human plasma was transferred into two prelabelled polypropylene tubes, containing approximately 1500 μL of plasma each (2 aliquots per time-point). Tubes were capped immediately from each time point and the plasma was frozen in an upright position at approximately −80° C. for storage.

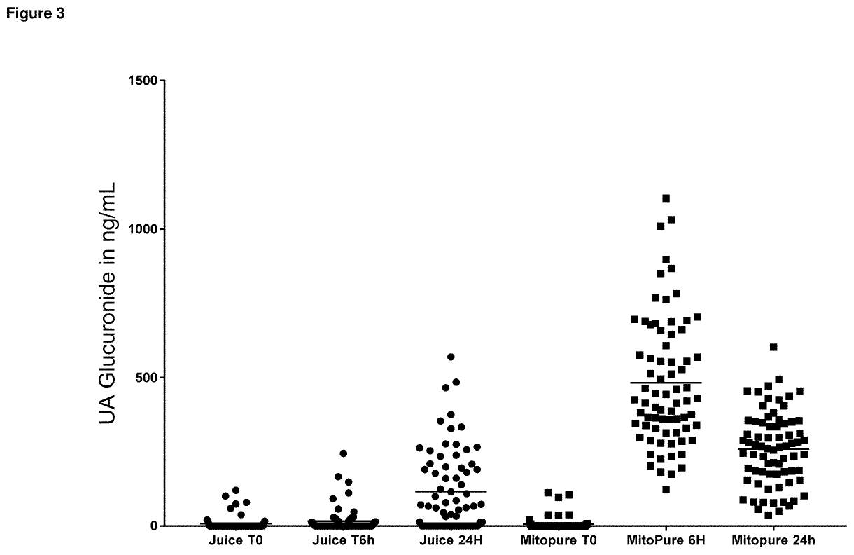

Plasma samples were collected to assess UA-glucuronide levels at the time points of 0 (baseline), 6 and 24 hrs. post intake after either Pomegranate juice or Urolithin A (Mitopure) in all n=100 healthy participants. Data is shown in FIG. 3 as is expressed as the mean of 100 samples.

### Dried Blood Spot (DBS) Collection:

The following items were used:
Blood collection card (filter paper—Whatman Filter Paper 903 5-spots card);
Sealable biohazard foil bags
Desiccant packs

Samples were collected by a finger-prick with a lancet. Each collection card was used for only one subjects. Blood was applied to the card with at least 3-4 blood spots on the DBS card. Care was taken to ensure the entire circle was uniformly saturated and each card. clearly labelled with appropriate identification. Follow the finger-prick with the lancet, gentle pressure was applied to the finger to allow a large drop of free-flowing blood to collect at the puncture site. The first drop was wiped away. Working quickly, the filter paper was held by the edges and the filter paper touched gently against the large drop of blood and in one step a sufficient quantity of blood was allowed to soak through and completely fill or saturate a circle. A completed saturated spot contained 20-40 μl of blood. Spotting was repeated, until enough blood was collected to fill at least 3 circles on the blood collection card. Completing filling of the blood spot circle was important as the laboratory used a hole puncher to punch a section of the circle of blood for testing. DBS samples were stored at room temperature in biohazard foil bags with clear labels

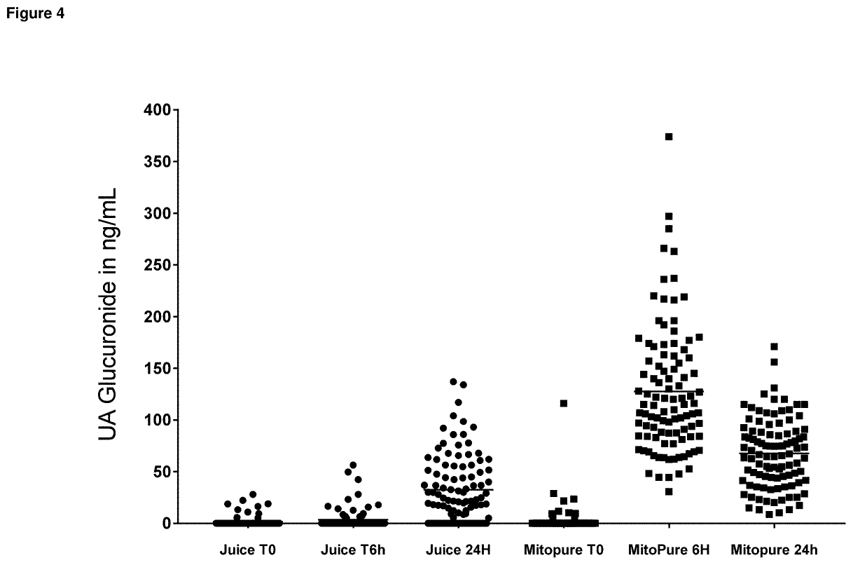

DBS (dried blood spot) samples were collected to assess UA-glucuronide levels at the timepoints of 0 (baseline), 6 and 24 hrs. post intake after either Pomegranate juice or Urolithin A (Mitopure) in all n=100 healthy participants. Data is shown in FIG. 4.

### UA Bioavailability Measurements

Measurements were performed according to the FDA Guidance for Industry and EMA guidelines on bioanalytical method validation as previously described (Toney et al (2019) Obesity (Silver Spring) 27(4):612-20). The quantification of UA and its metabolites—i.e. UA-glucuronide and UA-sulfate in plasma—was performed by column separation with reverse phase chromatography followed by detection with triple stage quadrupole MS/MS in the selected reaction monitoring mode. Plasma levels were assessed following oral intervention with either Mitopure or PJ at the T0, T6 and T24 hrs. time points for bioavailability assessments. The limit of quantification in plasma was 5.00 pg/mL for parent-UA and 5.00 ng/mL for both the UA-glucuronide and UA-sulfate its metabolites. An analytical method to determine UA-glucuronide in human whole blood collected in dried blood spots (DBS) using liquid chromatography coupled to mass spectrometry (LC MS/MS) was also developed and validated. The limit of quantification for UA-glucuronide in human dried blood spots samples (6-mm spots) range was 5.00 to 5000 ng/mL for the analysis of human dried blood spots samples (6-mm spots). The quantification of UA-glucuronide was performed by column separation with reversed phase liquid chromatography followed by detection with triple stage quadrupole MS/MS in the selected reaction monitoring mode.

### Results

The results show that in both plasma and DBS method that very few subjects (12%) with circulating UA-glucuronide at baseline from natural exposure to dietary precursors of Urolithin A.

Following dietary challenge with 100% pomegranate juice (rich in dietary ellagitannins), these levels did not change drastically at 6 hours post intake, but by 24 hours approx. 40% of subjects had metabolized the precursors and considerable levels of UA-glucuronide that were detected in a few drops of dried whole blood. These subjects were considered as “High producers”. The remaining 60% were either unable to convert (33%) or were poor convertors (27%). Subjects were categorized as low-producers with <100 ng/mL circulating urolithin A glucuronide levels in plasma or high-producers with ≥100 ng/mL circulating urolithin A glucuronide levels in plasma.

In comparison with consumption of PJ, with Urolithin A (Mitopure product), at baseline (after the washout period of 10-12 days) similar baseline levels were observed. Following oral administration of Urolithin A (Mitopure), within 6 hours all 100 participants had significantly higher concentration of UA-glucuronide (maximal concentration-Cmax). These levels came close to steady state levels at 24 hours with all subjects showing detectable levels of UA-glucuronide (levels with Urolithin A (Mitopure) were significantly higher in comparison to pomegranate juice >2 fold when comparing mean of population, p<0.0001 at both 6 and 24-hour time-points). Exposure to UA following intake (i.e. area under the curve-AUC) was >6-fold higher with Urolithin A (Mitopure) compared to 100% pomegranate juice.

TABLE 1
PK Parameters (Plasma Mean Concentrations)
on Plasma UA-Glucuronide levels Urollithin
A (Mitopure) Vs. 100% Pomegranate juice at
different time points of 0, 6 and 24 hrs.

| Product | Time (hours) | Mean | Std. Error |
|---|---|---|---|
| 100% Pomegranate Juice | T0 | 5.481 | 2.04 |
| | T6 | 12.840 | 17.026 |
| | T24 | 110.473 | 13.049 |
| UA Mitopure supplement | T0 | 9.568 | 4.862 |
| | T6 | 480.750 | 23.66 |
| | T24 | 255.525 | 12.943 |

TABLE 2
Table PK incremental AUC (area under the curve)
over time on Plasma UA-Glucuronide concentrations

| Product | Mean | Std. Error |
|---|---|---|
| 100% Pomegranate Juice | 68728.35 | 9188.13 |
| UA Mitopure supplement | 471857.30 | 20840.53 |

TABLE 3
Table PK Parameters (DBS Mean Concentrations)
on DBS UA-Glucuronide levels (Urolithin A
(Mitopure) Vs. 100% Pomegranate juice at
different time points of 0, 6 and 24 hrs.

| Product | Time | Mean | Std. Error |
|---|---|---|---|
| 100% Pomegranate Juice | T0 | 1.502 | .952 |
| | T6 | 3.521 | 4.452 |
| | T24 | 32.790 | 3.365 |
| UA Mitopure supplement | T0 | 2.423 | .948 |
| | T6 | 127.646 | 4.429 |
| | T24 | 66.866 | 3.348 |

### Comparison of the Levels of Urolithin a Glucuronide Measured in Plasma and Dry Blood Spots

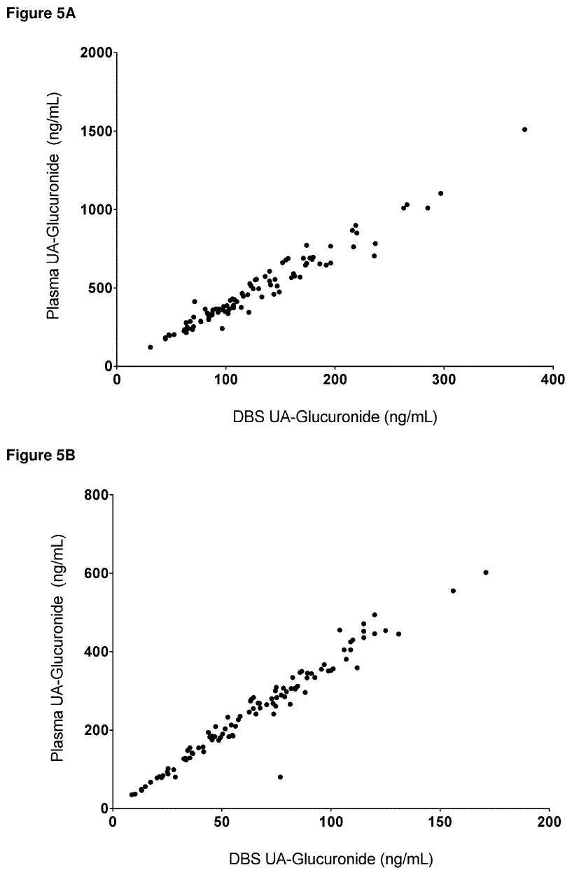

DBS levels of UA-Glucuronide were compared to plasma levels to see if the DBS method (20 ul of dried capillary whole blood spotted on a DBS card) had similar accurate sensitivity and specificity compared to the plasma collection (12 mL of venous blood). The results in FIG. 5A and FIG. 5B show a very high correlation between the two methods (Spearman correlation co-efficient r=0.975) at both the 6 hours (FIG. 5A) and 24 hours (FIG. 5B) collection timepoint following Urolithin A (Mitopure) intake. As such the DBS method can easily be utilized to replace the more invasive venous collection of plasma.

### Faecal Sample Collection

Each participant was also supplied with a stool sample collection kit (OMNIGene OMR-200, DNA Genotek, Kanata, ON, K2V 1C2, Canada) at their screening visit to collect a sample at home prior to study visit V2. Upon return to the study site, the stool samples were stored in a −80° C. freezer.

### Example 5A—Shotgun Metagenomic Sequencing and Analysis of Microbiome

DNA was extracted from ˜0.1 g aliquots of the faecal samples, collected in Example 5) using the NucleoSpin® 96 Soil kit (Macherey Nagel). A minimum of one negative control was included per batch of samples from the DNA extraction and throughout the laboratory process (including sequencing). A ZymoBIOMICS™ Microbial Community Standard (Zymo Research) was also included in the analysis as a positive (mock) control. Before sequencing, the quality of the DNA samples was evaluated using agarose gel electrophoresis, and the quantity of the DNA was evaluated by Qubit 2.0 fluorometer quantitation. The prepared DNA libraries were evaluated using Qubit 2.0 fluorometer quantitation and Agilent 2100 Bioanalyzer for the fragment size distribution. Quantitative real-time PCR (qPCR) was used to determine the concentration of the final library before sequencing. The library was sequenced using 2×150 bp paired-end sequencing on an Illumina platform. A total of 99 fecal samples were sequenced to an average depth of 19.9 M read pairs (IIlumina 2×150 PE) per sample. 96.5% of the high-quality microbiome reads from a sample were mapped to a reference human gut gene catalog, and an average of 200 metagenomic species (MGS) were detected per sample. For MGS abundance profiling, a set of 1273 metagenomic species (MGS), which have highly coherent abundance and base composition in a set of 1776 independent reference human gut samples were detected. The analysis is based on the metagenomic species concept (17). To taxonomically annotate the MGSs, all the catalog genes were blasted to the NCBI RefSeq genome database (2018 Oct. 1). To annotate at the various taxonomic ranks, different levels of similarity were required (95, 95, 85, 75, 65, 55, 50 and 45% for subspecies, species, genus, family, order, class, phylum, super kingdom, respectively) and a minimum of 80% sequence coverage. The percentage of genes of each MGS that mapped to each species were calculated, and species level taxonomy were assigned to an MGS if >75% of its genes could be annotated to a single species. For genus, family, order, class and phylum, 60, 50, 40, 30 and 25% consistency levels were used, respectively. Furthermore, at species and at genus level, the MGS was not assigned if another set of more than 10% of the genes belonged to a single alternative species/genus.

### Results

### Gut Microbiome Diversity and Richness Play an Essential Role in Defining UA-Producers Versus Non-Producers

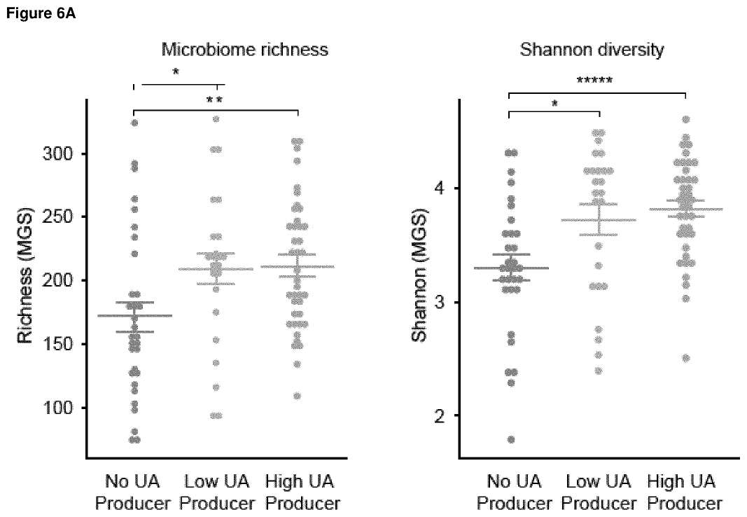

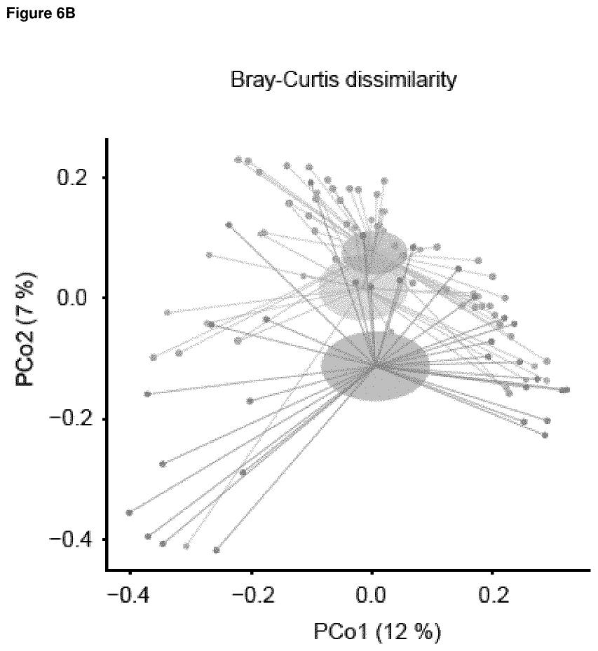

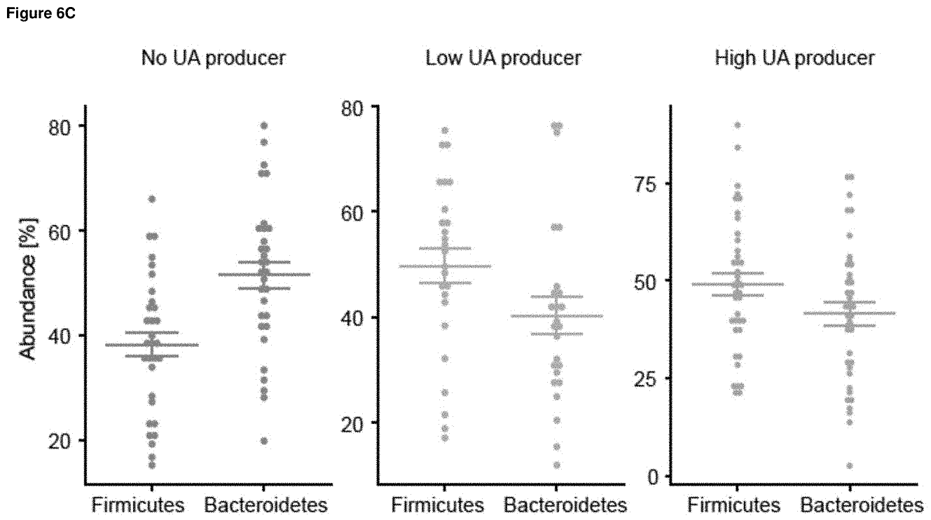

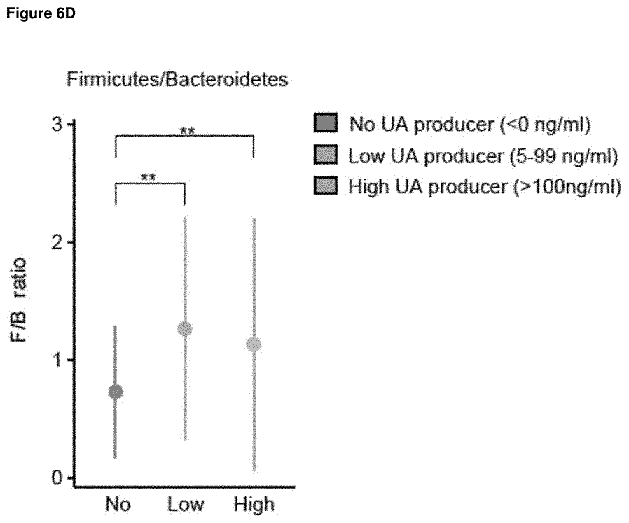

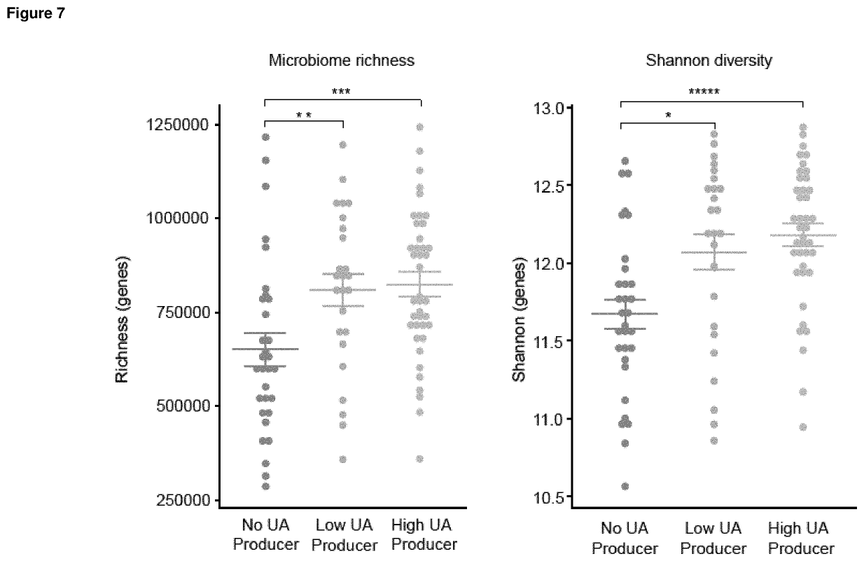

The microbiome composition from the faecal samples of individuals producing UA at different levels was investigated following pomegranate juice intake, using shotgun sequencing. This metagenomic analysis measured the abundance of both metagenomic species (MGS) and genes and compared them in subjects belonging to the no-, low- and high producer group. The metagenomic profiles were first used to determine the microbiome alpha diversity that indicates the variation of microbes in a single sample. Alpha diversity was assessed both as microbiome richness (number of species or genes observed in a sample) and microbiome variability, and was quantified using the Shannon index (18). The Shannon index accounts for the number of species or genes in a community, and also their relative abundance. A significantly higher richness was found in the microbiome Shannon index in both the low-producers and high-producers, when comparing each with the no-producer group, for both MGS (FIG. 6A) and gene-based (FIG. 7) measures. Secondly, changes in beta diversity was evaluated, which accounts for differences in relative abundance of MGSs among samples, using Bray-Curtis dissimilarity. Bray-Curtis dissimilarity can range between 0-1, where 0 means that the two samples have identical compositions (they share all species at the same relative abundance), and 1 means that the two samples are completely different (they do not share any species). A principal coordinate analysis (PCoA) of the Bray-Curtis dissimilarities (FIG. 6B) showed a striking shift in the overall microbiome composition when comparing non-producers with low (p=0.048) and high-producers (p=0.001), as shown by the clear segregation of the groups. No clear separation was observed between the groups of low-producer and high-producer. In summary, the alpha and beta diversity results indicate that the ability to convert UA from its precursors is significantly associated with a higher microbiome richness and overall composition.

### UA-Producer and Non-Producer Exhibit Differentially Abundant Gut Microbiome Taxa

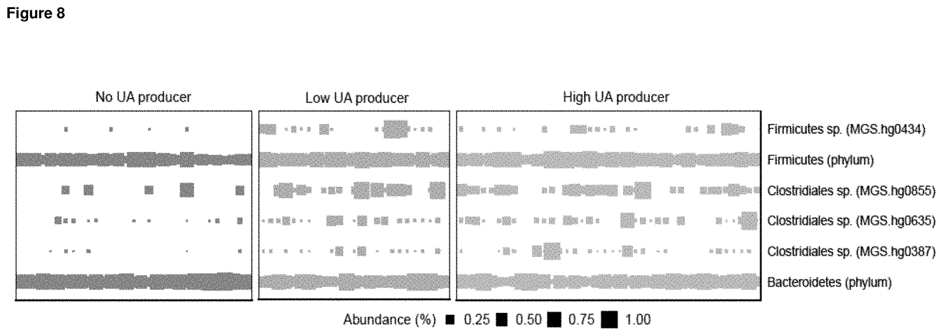

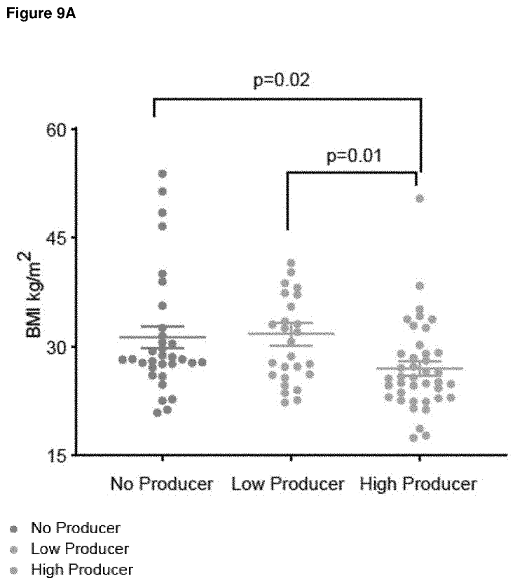

In line with the alpha and beta diversity results, we also found differentially abundant taxa when comparing UA producers (low and high production status) with non-producers. We first analyzed, at the phylum level, the abundancy of Firmicutes (F) and Bacteroidetes (B), as increased F/B ratio has been associated with several markers of gut and organismal health (Mariat et al (2009) BMC Microbiol 2009; 9:123; Wills et al (2014) PLoS One9(3):e90981; Verdam et al (2013) Obesity (Silver Spring) 21(12):E607-15). Both groups (low and high producers) capable of producing UA showed a higher abundance of Firmicutes with respect to Bacteroidetes, while the opposite was observed for the no-producer group (FIG. 6C). The F/B ratio was significantly higher in UA producers compared with non-producers (FIG. 6D). Next, we analyzed all changes at the levels of species (MGS) and of higher taxa among the three UA-producer status groups. We found 4 MGSs and 2 phyla with significantly different abundance or prevalence between the low-producer group and the no-producer group (FIG. 8). High-producers showed even larger differences, as 57 MGSs and 33 taxa (14 genera, 15 families, 4 phyla) had significantly different abundance or prevalence in the high-producer group compared with the no-producer group (Supplementary FIG. 6). Notably, all taxa that were significantly different between the no-producer and low-producer group were also significantly different between the no-producer and high-producer group.

### Physiological Differences Between UA Producers and Non Producers

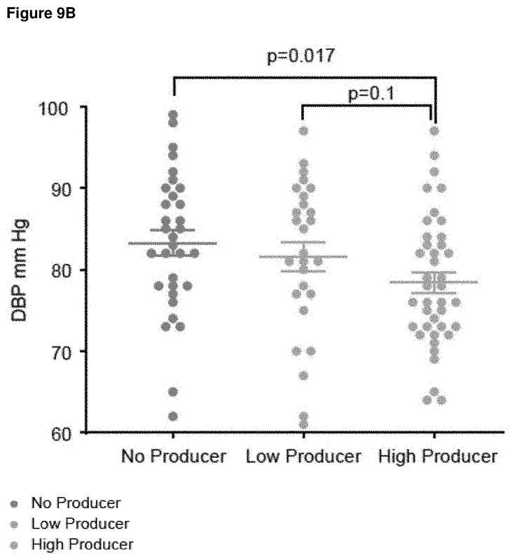

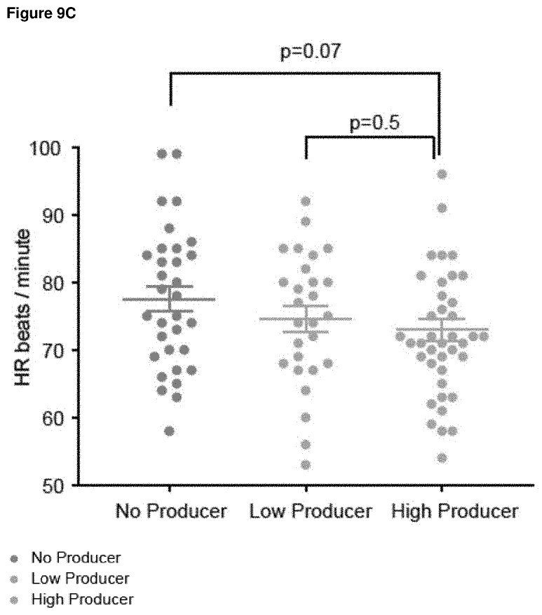

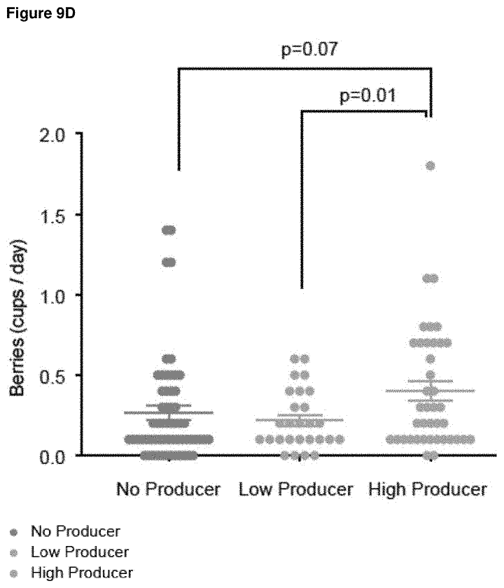

To better appreciate the potential impact of UA-producer status on general health, we performed a correlation analysis with participants' BMI and cardiac function (HR and DBP). One key observation was that high-producers had significantly lower BMI (27+6.1 kg/m2) compared with both low-producers (31.7+8 kg/m2; p=0.01) and non-producers (31.2+8.4 kg/m2; p=0.02) (FIG. 9A). The high-producers group also had significantly (p=0.017) lower mean resting diastolic blood pressure (78.4+8 mm Hg) compared with the non-producer's group (83.25+8.66 mm Hg) (FIG. 9B). Resting heart rate was also lower although not statistically significant in the high-producers compared with the non-producers (p=0.07) (FIG. 9C). There were no major differences in hematological and blood biochemistry parameters between the groups. The FFQ on dietary habits of the different groups revealed that the high-producer group consumed greater quantities of fruits and berries than the low and no-producer groups (FIG. 9D).

### Example 6: Measurement of Urolithin A and its Metabolites in Urine Samples

A single (Part A) and multiple (Part B) dose study of urolithin A was conducted to evaluate the safety, tolerability, pharmacokinetics and pharmacodynamics profile in healthy elderly subjects.

### Study Design

Part A: The study was a double-blind, randomized, single ascending doses, study in 24 healthy elderly male and female volunteers. Each subject was randomized for two subsequent doses in three cohorts.

Part B: The study was a double-blinded, randomized, multiple ascending dose study in 36 healthy elderly male and female volunteers. Each subject was randomised to receive study product or placebo for 28 days.

### Study Objectives:

To determine the safety and tolerability of urolithin A in healthy elderly subjects following multiple 28 days dosing.

To determine the pharmacokinetic profiles of urolithin A following a single and a multiple dose.

To compare the pharmacokinetic profiles of urolithin A delivered as soft gel formulation in a single 250 mg dose to ascending single higher doses administration at doses: 500 mg, 1000 mg and 2000 mg.

To compare the pharmacokinetic profiles of urolithin A delivered as a softgel formulation in repeated multiple 28 days 250 mg dose to ascending repeat multiple 28 days administration at doses: 500 mg and 1000 mg.

### Investigational Product:

1100 mg soft gel capsule containing 250 mg of urolithin A (as described in Example 2 above). Soft gel capsules were blister-packed in bulk, Labelling was in accordance with local regulatory specifications and requirements.

### Dose Per Intake:

Part A: 250 mg, 500 mg, 1000 mg or 2000 mg (1, 2, 4 or 8 capsules)

Part B: 250 mg per day, 500 mg per day or 1000 mg per day (1, 2, or 4 capsules per day)

### Placebo:

Soft gel capsule containing lecithin, triglycerides, diglycerides

### Timing For Intake:

Part A: Single oral dose administration on D1 of each period according to the randomisation. The administration took place around 8:00 am with around 200 mL tap water, in sitting position, and under fasting conditions.

Part B: Repeated oral dose administration from day 1 to day 28 according to the randomization. The administration took place around 8:00 am with around 200 mL tap water, in a sitting position, and under fasting conditions.

### Subjects:

### Part A:

24 healthy elderly male and female subjects were included in the study, within the age range 61 to 85 years.

Cohort 1 (8 subjects): 250 mg urolithin A (6 subjects) or placebo (2 subjects) capsule soft gel formulation then 2000 mg urolithin A or placebo capsule soft gel formulation. Cohort 2 (8 subjects): 500 mg urolithin A (6 subjects) or placebo (2 subjects) capsule soft gel formulation. Cohort 3 (8 subjects): 1000 mg urolithin A (6 subjects) or placebo (2 subjects) capsule soft gel formulation.

### Part B:

36 healthy elderly male and female subjects were included in the study, within the age range 61 to 85 years.

Cohort 1 (12 subjects): 250 mg urolithin A (9 subjects) or placebo (3 subjects) soft gel capsule formulation for 28 days.

Cohort 2 (12 subjects): 500 mg urolithin A (9 subjects) or placebo (3 subjects) soft gel capsule formulation for 28 days.

Cohort 3 (12 subjects): 1000 mg urolithin A (9 subjects) or placebo (3 subjects) soft gel capsule formulation for 28 days.

### Study Duration

### Part A:

Screening within 21 days prior to the first administration.

Hospitalization for 48h (D-1 evening to D2 evening) for each period.

Ambulatory visit at D4 and D5 for each period

Wash-out: at least 21 days between each administration

End of study visit: P2D5.

Follow up phone call at P2D7 (±2).

Expected duration: approximately 8 weeks for each participating subject

### Part B:

Screening within 21 days prior to the first administration

Ambulatory visits at day −1 (V1), day 7 (V2), day 14 (V3).

Hospitalisation from day 27 (V4) (around 4 pm) to day 29 (V6) (around 10 am)

Ambulatory visit at day 31 and day 32 for each period

Follow up phone call at day 35 (+/−2)

Expected duration: approximately 8 weeks for each participating subject.

During the last visit, subjects underwent a complete clinical biological examination, identical to an examination at the start of the study. Any (AEs) were recorded, and if they were ongoing a further follow-up was arranged. Follow up continued until the event was resolved or the condition was unlikely to change or the subject was lost to follow-up.

### Randomization

A randomisation list was provided by the sponsor's representative. The product was allocated at P1D1 for part A and on D-1 (V1) on part B.

### Blinding

The following measures were taken to avoid bias:
double-blind study; and
soft-gel capsules containing active product and placebo were indistinguishable in appearance.

The analytical centre as well as the Investigator and the team and the subject were in blind conditions. For each subject, a coding list containing the identification of the product (emergency envelopes) was supplied by the sponsor's representative and kept in a safe place during the whole clinical study period. In the case of a pharmaceutical preparation being required, the decoding system used was a sealed coding list to be given to the representative's pharmacist. The sealed coding list was kept in a safe place and was accessible to any person authorised to unblind.

### Pharmacokinetic Variables

### Urine

The percentage of an administered dose excreted from time zero to 36 h after dosing: Ae₀₋₃₆ₕ (Part A only)

The % of total dose excreted in urine (Part A only)

### Urine Collection

In Part A of the study, total urine was collected during the following intervals: 0-4 h, 4-8 h, 8-12 h, 12-24 h and 24-36 h post-dose. The collected urine volume of each collection period was carefully measured by weight and recorded in the eCRF, as well as the exact start and stop time of the collection interval. Each fraction of collected urine was homogenized and 2 aliquots of 5 mL per interval were kept and stored below −80° C. Following a protocol amendment to extend the blood sampling scheme from 36 to 96 h post-dose, additional spot urine samples were taken 72 and 96 h post-dose but urine was not collected quantitatively.

### Bioanalysis

The concentrations of Urolithin A and its metabolites in urine was determined using validated LC-MS/MS assays. The limit of quantification (LOQ) was 10 ng/mL for Urolithin A in urine and 50 ng/mL for Urolithin A glucuronide and Urolithin A sulphate in urine.

TABLE 4
shows the levels of Urolithin A glucuronide (ng/mL) in urine
for 250 mg, 500 mg, 1000 mg and 2000 mg up to 96 hours after
administration. Data is expressed as mean ± standard error.

| Time after | | | | |
|---|---|---|---|---|
| Urolithin A | Urolithin A dose (mg) | | | |
| administration | 250 | 500 | 1000 | 2000 |
| Predose | 4,114 ± 1,261 | 4,114 ± 1,261 | 4,114 ± 1,261 | 4,114 ± 1,261 |
| T0-T4 h | 8,536 ± 1,394 | 40,167 ± 6,802 | 70,492 ± 12,094 | 39,500 ± 6,033 |
| T4 h-T8 h | 31,617 ± 7,637 | 86,758 ± 12,051 | 76,417 ± 10,635 | 65,317 ± 10,853 |
| T8 h-T12 h | 40,453 ± 9,275 | 63,800 ± 9,043 | 64,967 ± 8,075 | 50,450 ± 5,793 |
| T12 h-T24 h | 62,817 ± 8,454 | 81,125 ± 10,792 | 107,983 ± 17,683 | 114,817 ± 12,123 |
| T24 h-T36 h | 33,962 ± 8,708 | 44,242 ± 4,631 | 59,575 ± 9,560 | 60,533 ± 12,216 |
| T72 h | N.D. | 15,706 ± 3,751 | 29,412 ± 5,717 | 70,567 ± 17,054 |
| T96 | N.D. | 8,720 ± 3,113 | 16,335 ± 3,897 | 38,487 ± 12,858 |

1. A method of determining whether a human subject will benefit from taking a urolithin supplement, comprising the steps of analysing a sample of a biological fluid collected from the subject, ascertaining whether the biological fluid contains a urolithin or a urolithin conjugate, then determining whether the subject is one who will benefit from ingesting a urolithin supplement from the level of the urolithin or urolithin conjugate in the biological fluid;
wherein the sample of biological fluid is selected from urine or blood and in the case of blood is analysed in the form of a dried blood spot.

2. The method of claim 1, wherein the urolithin conjugate is urolithin A glucuronide and the biological fluid is blood and wherein if the level of urolithin A glucuronide is under 100 ng/mL, then the subject is one who will benefit from a dose of a urolithin supplement.

3. The method of claim 1, the urolithin conjugate is urolithin A glucuronide and the biological fluid is urine and wherein if the level of urolithin A glucuronide is under 50,000 ng/mL, then the subject is one who will benefit from a dose of a urolithin supplement.

4. A method for determining a treatment dose of a urolithin supplement for a human subject, comprising
a) Measuring the level of a urolithin or a urolithin conjugate in a biological fluid; and
b) Determining the treatment dose of the urolithin supplement from the level of urolithin or urolithin conjugate in the biological fluid sample
wherein the sample of biological fluid is selected from urine or a dried blood spot.

5. The method of claim 4, wherein the urolithin conjugate is urolithin A glucuronide and the biological fluid is a dried blood spot and:
a) if the level of urolithin A glucuronide is less than 5 ng/mL then the treatment dose of urolithin supplement is 1500 mg or 1000 mg per day, for example, 1000 mg per day;
b) if the level of urolithin A glucuronide is between 5 ng/mL and 50 ng/mL then the treatment dose of urolithin supplement is 500 mg per day; and
c) if the level of urolithin A glucuronide is between 50 ng/mL and 100 ng/mL the treatment dose of urolithin supplement is 250 mg/day.

6. The method of claim 4, wherein the urolithin conjugate is urolithin A glucuronide and the biological fluid is urine and:
a) if the level of urolithin A glucuronide is less than 10,000 ng/mL then the treatment dose of urolithin supplement is 1500 mg or 1000 mg per day, for example, 1000 mg per day;
b) if the level of urolithin A glucuronide is between 10,000 ng/mL and 25,000 ng/mL then the treatment dose of urolithin supplement is 500 mg per day; and
c) if the level of urolithin A glucuronide is between 25,000 ng/mL and 50,000 ng/mL the treatment dose of urolithin supplement is 250 mg/day.

7. A method for determining whether a human subject will benefit from taking a urolithin supplement, comprising
a) Measuring the level of a urolithin or a urolithin conjugate in a first biological fluid sample, collected prior to administration of a test dose of a urolithin or a urolithin precursor;
b) Administering a test dose of a urolithin or a urolithin precursor, for example pomegranate juice;
c) Measuring the level of a urolithin or a urolithin conjugate in a second biological fluid sample; and
d) Determining whether the subject is one who will benefit from ingesting a urolithin supplement from the increase in level of urolithin or urolithin conjugate in the biological fluid sample;
wherein the sample of biological fluid is selected from urine or a dried blood spot.

8. The method of claim 7, wherein a urolithin is administered, the urolithin conjugate is urolithin A glucuronide and the biological fluid is a dried drug spot, and wherein if the level of urolithin glucuronide is above 5 ng/mL and under 100 ng/mL, then the subject is one who will benefit from a dose of a urolithin supplement.

9. The method of claim 7, wherein a urolithin precursor is administered, the urolithin conjugate is urolithin A glucuronide and the biological fluid is a dried drug spot, further comprising calculating the dose of the urolithin supplement.

10. The method of claim 7, wherein a urolithin is administered, the urolithin conjugate is urolithin A glucuronide and the biological fluid is urine, and wherein if the level of urolithin glucuronide is above 10,000 ng/mL and under 50,000 ng/mL, then the subject is one who will benefit from a dose of a urolithin supplement.

11. The method of claim 7, wherein a urolithin precursor is administered, the urolithin conjugate is urolithin A glucuronide and the biological fluid is urine, further comprising calculating the dose of the urolithin supplement.

12. The method of claim 7, wherein the biological fluid sample is collected either;
(a) between about 6 to about 8 hours after administration of the urolithin or urolithin precursor;
(b) between about 18 to about 30 hours after the administration of the urolithin or urolithin precursor; or
(c) at about 24 hours after the administration of the urolithin or the urolithin precursor.

13. The method of claim 7, wherein the test dose of urolithin is selected from 250 mg, 500 mg or 1000 mg.

14. The method of claim 1, wherein the conjugate is a urolithin glucuronide or a urolithin sulphate.

15. The method of claim 1, wherein the dose of urolithin supplement is selected from 250 mg per day, 500 mg per day or 1000 mg per day or 1500 mg per day.

16. A computer-implemented method comprising the steps of:
(a) Optionally receiving data confirming transit of sample of a biological fluid from a subject;
(b) Receiving data of the level of a urolithin or urolithin conjugate in the biological fluid;
(c) Optionally receiving subject data, for example, time and date, age, sex, weight, ethnicity, sample type etc.
(d) Comparing the level of urolithin or a urolithin conjugate with data correlating the level with a treatment dose of a urolithin, for example, urolithin A, to calculate the treatment;
(e) Optionally communicating results to the subject.

17. A non-transitory computer-readable medium comprising computer-executable instructions that, when executed by a processor of a computing device, cause the computing device to perform a method, the method comprising:
(f) Optionally receiving data confirming transit of sample of a biological fluid from a subject;
(g) Receiving data of the level of a urolithin or urolithin conjugate in the biological fluid;
(h) Optionally receiving subject data, for example, time and date, age, sex, weight, ethnicity, sample type etc.
(i) Comparing the level of urolithin or a urolithin conjugate with data correlating the level with a treatment dose of a urolithin, for example, urolithin A, to calculate the treatment dose;
(j) Optionally communicating results to the subject.

18. A non-transitory computer-readable medium comprising computer-executable instructions of claim 17, wherein the data correlating the level with the treatment dose is:
the urolithin conjugate is urolithin A glucuronide and the biological fluid is a dried blood spot and:
a) if the level of urolithin A glucuronide is less than 5 ng/mL then the treatment dose of urolithin supplement is 1500 mg or 1000 mg per day, for example, 1000 mg per day;
b) if the level of urolithin A glucuronide is between 5 ng/mL and 50 ng/mL then the treatment dose of urolithin supplement is 500 mg per day; and
c) if the level of urolithin A glucuronide is between 50 ng/mL and 100 ng/mL the treatment dose of urolithin supplement is 250 mg/day; or
the urolithin conjugate is urolithin A glucuronide and the biological fluid is urine and:
a) if the level of urolithin A glucuronide is less than 10,000 ng/mL then the treatment dose of urolithin supplement is 1500 mg or 1000 mg per day, for example, 1000 mg per day;
b) if the level of urolithin A glucuronide is between 10,000 ng/mL and 25,000 ng/mL then the treatment dose of urolithin supplement is 500 mg per day; and
c) if the level of urolithin A glucuronide is between 25,000 ng/mL and 50,000 ng/mL the treatment dose of urolithin supplement is 250 mg/day.

19. A computer device comprising a processor, memory and display, the computing device being arranged to implement a method of claim 16.

20. The method of claim 1, further comprising administering a urolithin supplement to the subject.

21. The method of claim 4, then further comprising administering a urolithin supplement to the subject.

22. A method of preventing or treating symptoms of aging in a human subject wherein the method comprises administering a urolithin supplement to the subject and wherein the subject is one who has been determined as benefiting from taking a urolithin supplement by a method of claim 1.

23. A method of maintaining adequate nutrient levels in a human subject wherein the method comprises administering a urolithin supplement to the subject and wherein the subject is one who has been determined as benefiting from taking a urolithin supplement by a method of claim 1.

24. A method of determining the health of an individual's gut microbiome comprising the steps of analysing a sample of biological fluid collected from the subject, ascertaining whether the biological fluid, contains a urolithin or a urolithin conjugate, as an indicator of the health of the individual's gut microbiome;
wherein the sample of biological fluid is selected from urine or blood and in the case of blood is analysed in the form of a dried blood spot.

25. The method of determining the health of an individual's gut microbiome of claim 24, further comprising the collecting a stool sample for microbiome analysis and analysing the stool sample.
